(19) United States
(12) Patent Application Publication
(10) Pub. No.: US 2025/0215463 A1
(43) Pub. Date: Jul. 3, 2025

(54) COMPOSITIONS COMPRISING MILK FAT TRIGLYCERIDES PRODUCED BY MICROBIAL FERMENTATION

(71) Applicant: Manus Bio Inc., Waltham, MA (US)

(72) Inventors: Arthur J. SHAW, IV, Waltham, MA (US); Yannick Remi BRUNET, Waltham, MA (US); Ajikumar PARAYIL KUMARAN, Waltham, MA (US); Christine Nicole S. SANTOS, Waltham, MA (US)

(21) Appl. No.: 18/843,862

(22) PCT Filed: Mar. 6, 2023
(86) PCT No.: PCT/US2023/063780

§ 371 Date: Sep. 4, 2024

Related U.S. Application Data
(60) Provisional application No. 63/316,521, filed on Mar. 4, 2022.

(51) Int. Cl. C12P 7/6463; A23D 9/02; C12N 1/20; C12N 9/00; C12N 9/10; C12N 9/90

(52) U.S. Cl. CPC C12P 7/6463; A23D 9/02; C12N 1/20; C12N 9/1029; C12N 9/90; C12N 9/93; C12Y 203/0102; C12Y 203/01085; C12Y 502/01005; C12Y 602/01003

(57) ABSTRACT

The present disclosure relates to microbial hosts and methods for biosynthesis of compositions comprising milk fat triglycerides.

### PRIORITY

This application claims the benefit of, and priority to, U.S. provisional application No. 63/316,521 filed Mar. 4, 2022, which is hereby incorporated by reference in its entirety.

### BACKGROUND

Dairy milk and dairy milk products have been consumed around the world for millennia. However, due to the health issues such as lactose intolerance and milk allergy; ethical issues such as poor treatment of animals; as well as environmental issues such as land use, water use, and greenhouse gas emission, there has been an increased demand for alternative milks and milk products around the world. Alternatives coming from plant sources, such as soy milk, almond milk, oat milk, rice milk, cashew milk and coconut milk are heavily available as non-dairy alternatives. However, apart from nutritional differences from dairy milk, the non-dairy alternatives suffer from non-milk tastes and flavors (e.g., beany flavor or nutty flavor). Because milk triglyceride (fat) has unique chemical properties, plant oils cannot match the physical and sensory properties of dairy milk. Therefore, sustainable and scalable processes for biosynthesizing compositions that have similar chemical and sensory properties to dairy milk fat are desirable.

### SUMMARY OF THE DISCLOSURE

In various aspects, the present disclosure provides methods for making compositions that are chemically similar to milk fat (e.g., dairy milk fat), and provides host cells for use in these methods.

Accordingly, in one aspect, the present disclosure provides engineered host cells (e.g., microbial host cells) for producing triglycerides characteristic of dairy milk by microbial fermentation or bioconversion. In some embodiments, the composition further comprises one or more lactones that are characteristic of milk fat, to provide a desired sensory profile. In some embodiments, the composition further comprises β-carotene and vitamin A, which among other things, provides a color of the composition more similar to dairy milk products. The present disclosure further provides methods of making products containing the composition, including milk, cheese, and butter, among other products that typically involve dairy. Such milk fat-containing products can be made at reduced cost and more sustainable fashion by virtue of this disclosure.

Accordingly, in some aspects the invention provides a microbial cell for producing milk fat triglycerides. The microbial cell expresses a biosynthetic pathway comprising at least one heterologous enzyme, where the biosynthetic pathway produces triglycerides having short chain fatty acids esterified at sn-3. For example, the cell produces triglycerides having at least about 5% C4 and C6 fatty acids esterified at sn-3 on a molar basis. In various embodiments, the cell produces triglycerides where about 25% to about 75% of fatty acids esterified at sn-3 (on a molar basis) are C4 and C6 fatty acids. The triglycerides will further comprise C12 to C18 fatty acids esterified at sn-3. In various embodiments, the triglycerides comprise C4:0, C6:0, C8:0, and C10:0 fatty acids esterified at sn-3.

In various embodiments, the triglycerides have predominately C12 to C18 fatty acids esterified at sn-1 and sn-2 (i.e., on a molar basis). For example, in some embodiments, the triglycerides comprise 12:0, 14:0. 16.0, 16:1, 18:0, 18:1, and 18:2 fatty acids esterified at sn-1. In some embodiments, the triglycerides comprise 12:0, 14:0. 16.0, 16:1, 18:0, 18:1, and 18:2 fatty acids esterified at sn-2. In some embodiments, the triglycerides comprise linoleic acid esterified at sn-2.

In some embodiments, the biosynthetic pathway producing the triglycerides comprises at least one heterologous fatty acyl-CoA synthetase (ACS) having specificity for short chain fatty acid substrates. In some embodiments, the biosynthetic pathway comprises at least one or at least two such heterologous ACS enzymes, which are optionally enzymes comprising an amino acid sequence that is at least 70% identical to an amino acid sequence selected from SEQ ID NOs: 1-7.

In some embodiments, the biosynthetic pathway producing the triglycerides comprises a heterologous diacylglycerol O-acyltransferase (DGA) having specificity for short chain fatty acid CoA substrate. In some embodiments, the DGA comprises an amino acid sequence that is at least about 70% identical to an amino acid sequence selected from SEQ ID NOs: 8-17.

In some embodiments, the microbial cell expresses a conjugated linoleic acid (CLA) isomerase. In some embodiments, the CLA isomerase comprises an amino acid sequence that is at least about 75% identical to an amino acid sequence selected from SEQ ID NO: 19.

In some embodiments, the microbial cell comprises a modification that results in decreased amount or activity of one or more endogenous diglyceride acyltransferases. In some embodiments, the microbial cell comprises a deletion(s) of genes encoding one or more endogenous diglyceride acyltransferases. For example, in some embodiments, the microbial cell has a deletion, inactivation, or reduced expression of one or more of DGA1 and DGA2 (Y. lipolytica) or ortholog thereof.

In some embodiments, the microbial cell has a deletion, inactivation, or reduced expression or activity of one or more fatty acid desaturase or fatty acid elongase enzymes. In such embodiments, the composition of the triacylglycerides can be further tuned.

In some embodiments, the microbial cell further comprises a biosynthetic pathway producing one or more lactones. In some embodiments, the microbial cell expresses one or more enzymes having fatty acid hydroxylase activity. In some embodiments, the enzyme is a cytochrome P450 enzyme (such as a CYP505 enzyme or derivative thereof). In certain embodiments, the enzymes described herein are engineered for hydroxylase activity at the desired position of a desired fatty acid substrate, to allow for production of the desired lactone. In some embodiments, the cell further expresses a cytochrome P450 reductase. In some embodiments, the P450 enzyme comprises a domain having fatty acid hydroxylase activity and CPR domain.

In some embodiments, the cell further produces β-carotene through a heterologous biosynthetic pathway, which can provide desirable color attributes for products such as butter and others. For example, the microbial cell can express heterologous biosynthetic enzymes such as a phytoene desaturase and/or a bifunctional lycopene cyclase/phytoene synthase. In some embodiments, the cell further produces vitamin A (e.g., from β-carotene substrate). In some embodiments, the microbial cell expresses a β-carotene 15,15′-monooxygenase, a retinal dehydrogenase and/or a lecithin: retinol acyltransferase. Precursors for β-carotene and vitamin A will be supplied by the mevalonic acid pathway (MVA) or non-mevalonic acid pathway (MEP pathway), which can be complemented or engineered for improved productivity. The MVA and MEP pathways produce iso-pentenyl pyrophosphate (IPP) and dimethylallyl pyrophosphate (DMAPP) precursors, which can be converted to farnesyl diphosphate.

In certain embodiments, fatty acids and/or fatty acid esters are fed to the cells to impact the fatty acid composition of triacylglycerides, including the composition of short and/or medium chain fatty acids, as well as saturated fatty acid composition, of the triacylglycerides. This allows the microbial cell to approach the desired fatty acid profile. For example, in some embodiments, the microbial cell is fed one or a combination of C4:0, C6:0, C8:0, C10:0, C12:0, C14:0, and C16:0.

In some embodiments, the microbial cell is a yeast or fungal cell. In some embodiments, the yeast or fungal cell is Yarrowia lipolytica. In some embodiments, the yeast or fungal cell is Yarrowia phangngensis. In some embodiments, the microbial cell is a bacterial cell. In some embodiments, the microbial cell is a bacterium that accumulates significant quantities of triacylglycerols.

In one aspect, the present disclosure relates to a method for making a composition comprising milk fat triglycerides. In some embodiments, the method comprises culturing a microbial cell according to any of the embodiments disclosed herein in the presence of fatty acid substrates. In some embodiments, the fatty acid substrates comprise one or a combination of C4:0, C6:0, C8:0, C10:0, C12:0, and C14:0 fatty acid substrates. The composition comprising milk fat triglycerides can be recovered from the culture. In some embodiments, the fatty acid substrates are added to the culture, optionally as alkyl esters or glycerides. In some embodiments, the fatty acid substrates are synthesized by the cell. In some embodiments, the composition is recovered by separating the wet cell mass, and purifying the compositions from the wet cell mass. In some embodiments, the cells are mechanically or enzymatically disrupted. The host cells and methods are further suitable for commercial production of the composition, that is, the cells and methods can be productive at commercial scale.

In another aspect, the present disclosure provides a composition or product (e.g., a beverage or food product) comprising milk fat triglycerides made according to the present disclosure.

In another aspect, the disclosure provides methods for making a product comprising milk fat triglycerides. The method comprises incorporating the composition comprising milk fat triglycerides of the instant disclosure into a product (i.e., a food, beverage, flavor, or food additive product). Examples of products in which the compositions may be used include, but are not limited to, food products, beverages, flavors, and food additives. Exemplary products include milk, cheese, butter, yogurt, frozen yogurt, gelato, milk chocolate, cream (e.g., heavy cream, light cream, sour cream, etc.), ice cream, cream cheese, custard, anhydrous milk fat, condensed milk, milk powder, and evaporated milk.

Other aspects and embodiments of the invention will be apparent from the following detailed description.

### BRIEF DESCRIPTION OF THE DRAWINGS

FIG. 1A to FIG. 1C show the composition of cow milk. FIG. 1A is a pie chart showing the composition of whole milk. FIG. 1B shows the primary classes of lipids present in cow milk. FIG. 1C shows the types of proteins present in cow milk.

FIG. 2A and FIG. 2B show the fatty acid composition and sn-position in dairy cow milk triglycerides. FIG. 2A illustrates major fatty acids in dairy cow milk triglycerides and vegetable oils. Key fatty acids for milk fat triglycerides are highlighted using squares. FIG. 2B shows the positional distribution of major fatty acids in dairy cow milk triglycerides.

FIG. 3A to FIG. 3C show a comparison of dairy cow milk triglycerides with plant triglycerides. FIG. 3A shows the structure of a representative milk triglyceride. FIG. 3B shows the structure of a typical plant triglyceride from plant oil (e.g., palm, soy, corn, sunflower, and safflower). FIG. 3C shows the structure of a representative medium chain oil (e.g., coconut and palm kernel).

FIG. 4A shows a schematic for metabolic engineering of yeast cells for the production of milk fat compositions. Carbon source such as sugar and fatty acids (e.g., 4:0, 6:08:0, 10:0, and 12:0 fatty acids) are used as substrates (which can be added to the culture). Carbon source(s) (e.g., sucrose, glucose, sugar cane, glycerol, starch, acetic acid) is/are converted to acetyl-CoA and malonyl-CoA through glycolysis and TCA cycle. 14:0-CoA, 16:0-CoA, 18:0-COA, 18:1-CoA and 18:2-acyl-phospholipid (PL) are synthesized by native fatty acid synthesis followed by successive action of elongase, 49 desaturase, and 412 desaturase. These fatty acyl-esters are converted to the indicated lysophosphatidic acid via the action of a glycerol 3-phosphate sn-1 acyltransferase (SCT), which is converted to diacylglycerol via the action of 1-acyl-sn-glycerol-3-phosphate acyltransferase (SLC) and a phosphatidate phosphatase (PAH). Free linoleic acid released from phospholipids via a phospholipase can be further converted to conjugated linoleic acid (CLA) via the activity of a heterologous conjugated linoleic acid isomerase and re-incorporated into the phospholipid and acyl-ester pools. The 8:0, 10:0, and 12:0 fatty acids can be converted to lactones, such as δ-dodecalactone, δ-decalactone, γ-dodecalactone, γ-decalactone, and δ-octalactone via the activity of a cytochrome P450 hydroxylase. The 4:0, 6:0, 8:0, 10:0, and 12:0 fatty acids are also converted to 4:0-CoA, 6:0-CoA, 8:0-COA, 10:0-CoA, and 12:0-CoA, which are incorporated into the sn-3 triglyceride position via a specialized diglycerol acyltransferase to generate milk fat triglycerides having the formula shown in a rectangle at bottom right. Finally, vitamin A and β-carotene can be biosynthesized by central metabolism via a farnesyl-pyrophosphate intermediate (See FIG. 4B).

FIG. 4B shows a schematic pathway for β-carotene and vitamin A (retinol and retinyl esters) biosynthesis. ERG10, acetyl-CoA C-acetyltransferase, EC 2.3.1.9; ERG13, 3-hydroxy-3-methylglutaryl-CoA (HMG-COA) synthase, EC 2.3.3.10; HMG1/HMG2, hydroxymethylglutaryl-CoA (HMG-COA) reductase, EC 1.1.1.88; ERG12, mevalonate kinase, EC 2.7.1.36; ERG8, phosphomevalonate kinase, EC 2.7.4.2; IDI1, isopentenyl diphosphate:dimethylallyl diphosphate isomerase, EC 5.3.3.2; BTS1/CrtE, geranylgeranyl diphosphate synthase, EC 2.5.1.1; CrtYB/CarRA/CarRP, Bifunctional lycopene cyclase/phytoene synthase, EC 2.5.1.32; CrtI/CarB, Phytoene desaturase, EC 1.3.99.31; BMCO, β-carotene 15,15′-monooxygenase, EC 1.13.11.63; RDH, Retinal dehydrogenase, EC 1.2.1.36; LRAT, Lecithin: retinol acyltransferase, EC 2.3.1.135. The following heterologous enzymes are expressed in Yarrowia lipolytica: CrtYB/CarRA/CarRP, CrtI/CarB, BMCO, RDH, and LRAT to enable the production of β-carotene and vitamin A.

FIG. 5 shows microscopy images of Y. lipolytica expressing heterologous DGAT enzymes.

FIG. 6 is a GC-MS spectra showing peak corresponding to hexanoic acid methyl ester present in MY74 lipid and absent in MY27 lipid.

FIG. 7A to FIG. 7D show downstream separation processes for the purification of milk fat compositions produced intracellularly in microbes.

### DETAILED DESCRIPTION

In various aspects, the present disclosure provides methods for making compositions that are chemically similar to milk fat (e.g., dairy milk fat), and provides host cells for use in these methods.

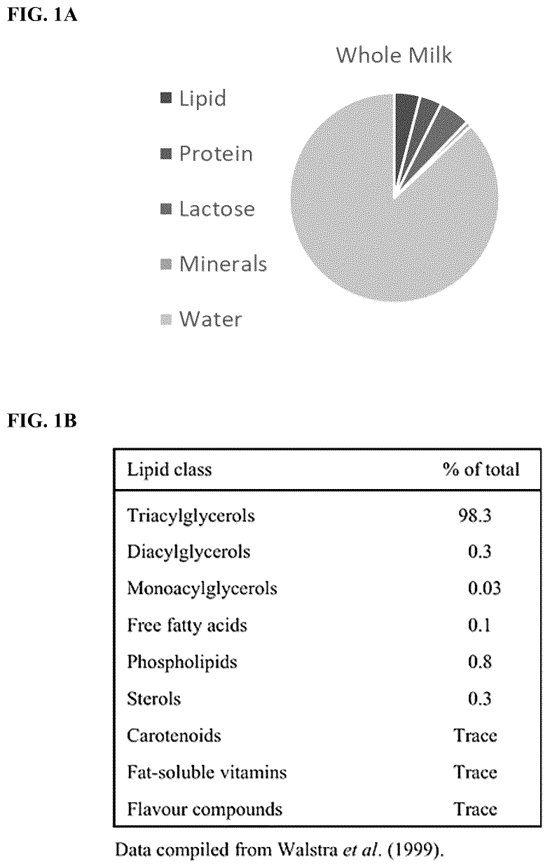

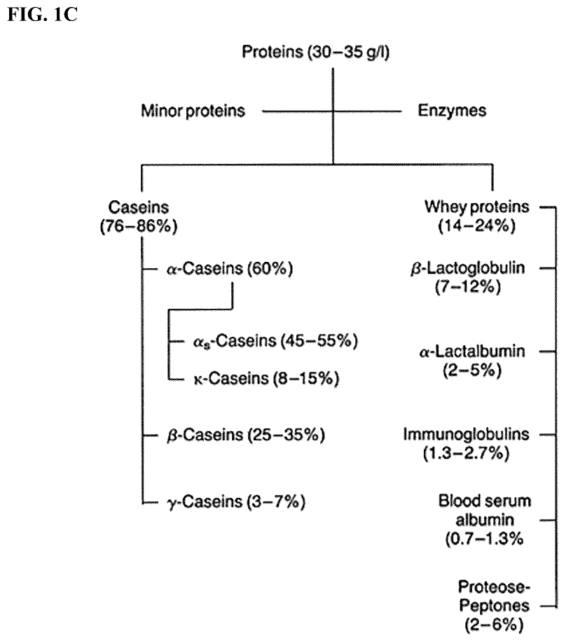

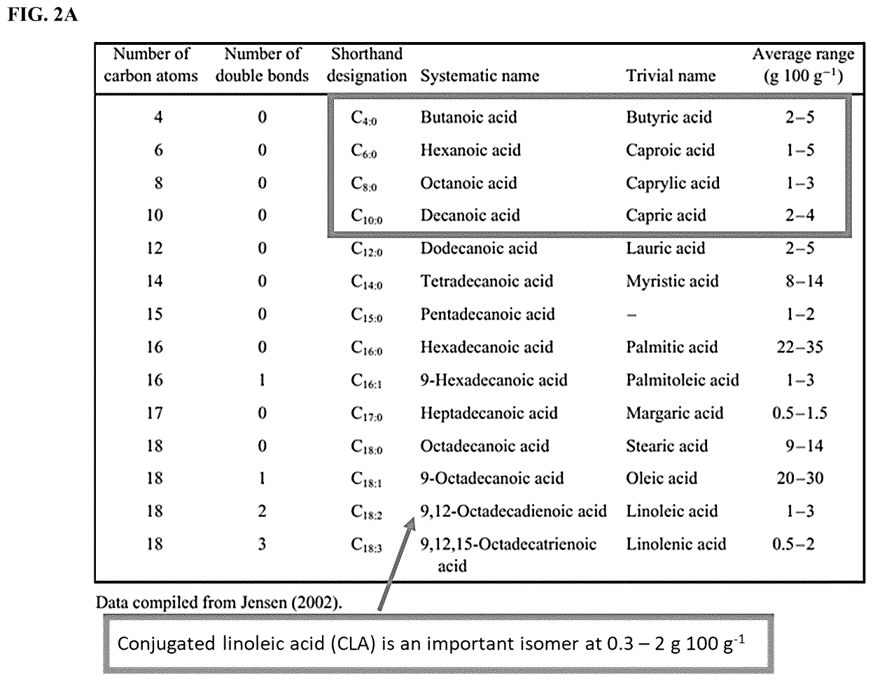

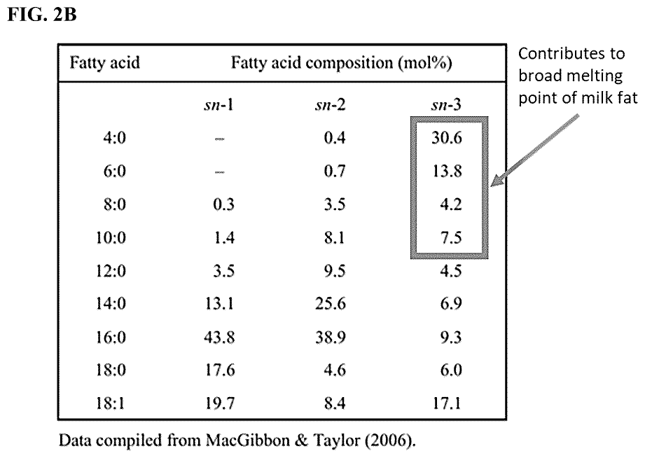

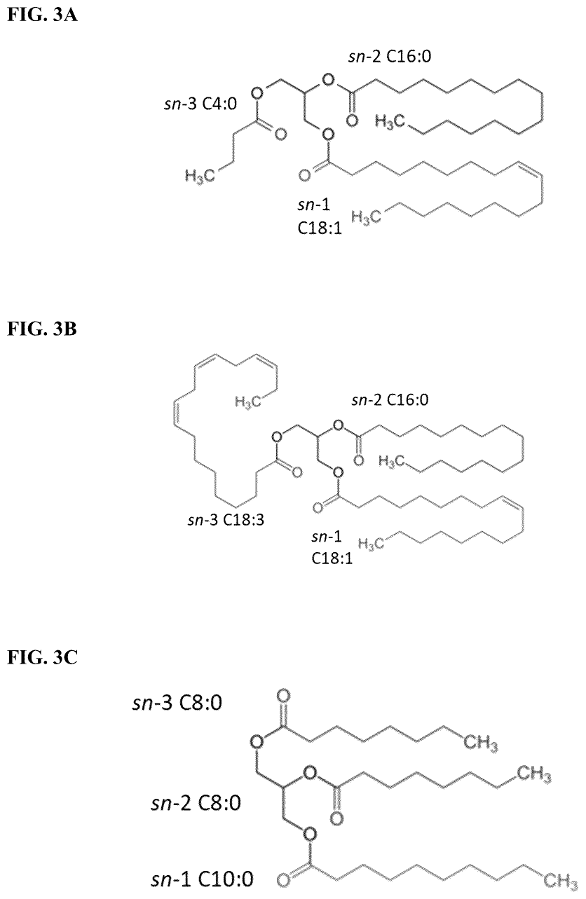

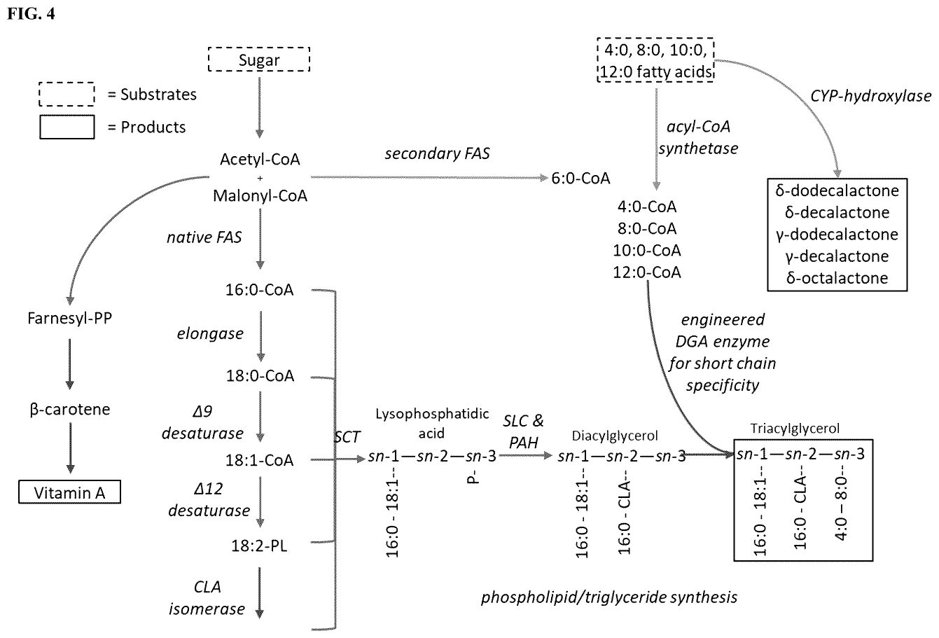

Dairy milk is an oil-in-water emulsion containing the macro components as shown in FIG. 1A, although the actual composition of milk varies by mammalian species, breed, season, nutrition, and other factors. Over 98% of the milk lipids can be triacylglycerides (FIG. 1B). Unlike vegetable oils, milk triglycerides contain low molecular weight fatty acids. FIG. 2A and FIG. 2B illustrate the major differences between vegetable oils and dairy milk triglycerides. Interestingly, the low molecular weight fatty acids are not uniformly distributed in milk triglycerides. As shown in FIG. 2B, unlike the sn-1 and sn-2 positions, which contains medium and long chain fatty acids, the sn-3 position has lower molecular weight fatty acids such as butyric acid and caproic acid. The most common fatty acids at sn-1 are C16:0, C18:1 and C18:0, while the most common fatty acids at sn-2 are C16:0, C14:0, and C12:0 (FIG. 2B and FIG. 3A).

Accordingly, in one aspect, the present disclosure provides engineered host cells (e.g., microbial host cells) for producing triglycerides characteristic of dairy milk (e.g., cow milk, sheep milk, goat milk, or buffalo milk) by microbial fermentation or bioconversion. In some embodiments, the composition further comprises one or more lactones that are characteristic of milk fat, to provide a desired sensory profile. In some embodiments, the lactones comprise one or more of δ-dodecalactone, δ-decalactone, γ-dodecalactone, γ-decalactone, and δ-octalactone. In some embodiments, the composition further comprises β-carotene and vitamin A, which among other things, provides a color of the composition more similar to dairy milk products. The present disclosure further provides methods of making products containing the composition, including milk, cheese, and butter, among other products that typically involve dairy. Such milk fat-containing products can be made at reduced cost and more sustainable fashion by virtue of this disclosure.

Accordingly, in some aspects the invention provides a microbial cell for producing milk fat triglycerides. The microbial cell expresses a biosynthetic pathway comprising at least one heterologous enzyme, where the biosynthetic pathway produces triglycerides having short chain fatty acids esterified at sn-3. For example, the cell produces triglycerides having at least about 5% C4 and C6 fatty acids esterified at sn-3 on a molar basis. In some embodiments, the cell produces triglycerides having at least about 7%, or at least about 10%, or at least about 15%, or at least about 20%, or at least about 25%, or at least about 30%, or at least about 35%, or at least about 40% C4 and C6 fatty acids esterified at sn-3 on a molar basis. In some embodiments, the cell produces triglycerides having at least about 45% or at least about 50% C4 and C6 fatty acids esterified at sn-3 on a molar basis. In various embodiments, the cell produces triglycerides where about 25% to about 75% of fatty acids esterified at sn-3 (on a molar basis) are C4 and C6 fatty acids (e.g., about 30% to about 70%, or about 40% to about 60%).

The triglycerides will further comprise C12 to C18 fatty acids esterified at sn-3. In various embodiments, the triglycerides comprise C4:0, C6:0, C8:0, and C10:0 fatty acids esterified at sn-3.

In various embodiments, the triglycerides have predominately C12 to C18 fatty acids esterified at sn-1 and sn-2 (i.e., on a molar basis). For example, in some embodiments, the triglycerides comprise 12:0, 14:0. 16.0, 16:1, 18:0, 18:1, and 18:2 fatty acids esterified at sn-1. In some embodiments, the triglycerides comprise 12:0, 14:0. 16.0, 16:1, 18:0, 18:1, and 18:2 fatty acids esterified at sn-2. In some embodiments, the triglycerides comprise linoleic acid esterified at sn-2.

In some embodiments, the biosynthetic pathway producing the triglycerides comprises at least one heterologous fatty acyl-CoA synthetase (ACS) having specificity for short chain fatty acid substrates. In some embodiments, the biosynthetic pathway comprises at least one or at least two such heterologous ACS enzymes, which are optionally enzymes comprising an amino acid sequence that is at least 70% identical to an amino acid sequence selected from SEQ ID NOs: 1-7. In some embodiments, the ACS enzyme(s) comprise an amino acid sequence that is at least about 75%, or at least about 80%, or at least about 85% or at least about 90% or at least about 95%, at least about 96%, at least about 97%, at least about 98%, or at least about 99% identical to an amino acid sequence selected from SEQ ID NOs: 1-7.

In some embodiments, the microbial cell expresses a heterologous ACS comprising an amino acid sequence that is at least about 75%, or at least about 80%, or at least about 85%, or at least about 90%, or at least about 95%, or at least about 96%, or at least about 97%, or at least about 98%, or at least about 99% identical to the amino acid sequence of SEQ ID NO: 2. In some embodiments, the microbial cell expresses (or further expresses) a heterologous ACS comprising an amino acid sequence that is at least about 75%, or at least about 80%, or at least about 85%, or at least about 90%, or at least about 95%, or at least about 96%, or at least about 97%, or at least about 98%, or at least about 99% identical to the amino acid sequence of SEQ ID NO: 3.

In some embodiments, the biosynthetic pathway producing the triglycerides comprises a heterologous diacylglycerol O-acyltransferase (DGA) having specificity for short chain fatty acid CoA substrate. In some embodiments, the DGA comprises an amino acid sequence that is at least about 70% identical to an amino acid sequence selected from SEQ ID NOs: 8-17. In some embodiments, the DGA comprises an amino acid sequence that is at least about 75%, or at least about 80%, or at least about 85%, or at least about 90%, or at least about 95%, or at least about 96%, or at least about 97%, or at least about 98%, or at least about 99% identical to an amino acid sequence selected from SEQ ID NOs: 8-17.

In some embodiments, the DGA comprises an amino acid sequence that is at least about 70% identical to the amino acid sequence SEQ ID NO: 8. In some embodiments, the DGA comprises an amino acid sequence that is at least about 75%, or at least about 80%, or at least about 85%, or at least about 90%, or at least about 95%, or at least about 96%, or at least about 97%, or at least about 98%, or at least about 99% identical to the amino acid sequence of SEQ ID NO: 8.

In some embodiments, the microbial cell expresses a conjugated linoleic acid (CLA) isomerase. In some embodiments, the CLA isomerase comprises an amino acid sequence that is at least about 75%, or at least about 80%, or at least about 85%, or at least about 90%, or at least about 95%, or at least about 96%, or at least about 97%, or at least about 98%, or at least about 99% identical to an amino acid sequence selected from SEQ ID NO: 19.

In some embodiments, the microbial cell comprises a modification that results in decreased amount or activity of one or more endogenous diglyceride acyltransferases. In some embodiments, the microbial cell comprises a deletion(s) of genes encoding one or more endogenous diglyceride acyltransferases. For example, in some embodiments, the microbial cell has a deletion, inactivation, or reduced expression of one or more of DGA1 and DGA2 (Y. lipolytica) or ortholog thereof.

In some embodiments, the host cell has one or more genetic modifications that reduce C16:1 fatty acid production and/or increase C18:1 fatty acid production. In some embodiments, the microbial cell has a deletion, inactivation, or reduced expression or activity of one or more fatty acid desaturase enzymes. In some embodiments, the microbial cell has a deletion, inactivation, or reduced expression or activity of a delta-12 desaturase (e.g., FAD2 delta-12 desaturase of Y. lipolytica or ortholog thereof). Alternatively or in addition, the microbial cell has a deletion, inactivation, or reduced expression or activity of one or more delta-9 desaturase enzymes (OLE1, YALI0C05951g or ortholog thereof). Alternatively or in addition, the microbial cell expresses a heterologous OLE1 converting C18:0 to C18:1. See, Sitepu IR. et al., Manipulation of culture conditions alters lipid content and fatty acid profiles of a wide variety of known and new oleaginous yeast species, Bioresource Technology, Vol. 144, 2013, 360-369. Such heterologous OLE1 may be selected from those that comprise an amino acid sequence having at least 80% sequence identity, or at least 85% sequence identity, or at least 90% sequence identity, or at least 95% sequence identity, or at least 97% sequence identity, or at least 98% sequence identity, or at least 99% sequence identity (or 100% sequence identity) to one of SEQ ID NOS: 22 to 30.

In some embodiments, the microbial cell has a deletion, inactivation, or reduced expression or activity of one or more fatty acid elongase enzymes. In some embodiments, the microbial cell has a deletion, inactivation, or reduced expression or activity of ELO1 (e.g., Y. lipolytica YALI0F06754g, or ortholog thereof). Alternatively or in addition, the microbial cell has a deletion, inactivation, or reduced expression or activity of ELO2 (e.g., YALI0F06754g, or ortholog thereof).

In some embodiments, the microbial cell expresses one or more heterologous fatty acid synthases. For example, the heterologous fatty acid synthase may produce C10:0, C12:0, and C14:0 fatty acids. In some embodiments, at least one heterologous fatty acid synthase comprises a subunit comprising an amino acid sequence that is at least 70%, or at least 80%, or at least 85%, or at least 90%, or at least 95%, or at least 97%, or at least 98%, or at least 99% identical (or has 100% sequence identity) to the amino acid sequence of SEQ ID NO: 20 (FAS1). In some embodiments, at least one heterologous fatty acid synthase comprises a subunit comprising an amino acid sequence that is at least 70%, or at least 80%, or at least 85%, or at least 90%, or at least 95%, or at least 97%, or at least 98%, or at least 99% identical (or has 100% sequence identity) to the amino acid sequence of SEQ ID NO: 21 (FAS2). An enzyme comprising these FAS1 and FAS2 subunits produces C10:0 fatty acids. In some embodiments, the microbial cell expresses a modified Y. lipolytica FAS1 and/or FAS2 as described Xu P. et al., Engineering Yarrowia lipolytica as a platform for synthesis of drop-in transportation fuels and oleochemicals, PNAS, 2016, vol. 113, no. 39, 10848-10853; Rigouin C., et al., Production of Medium Chain Fatty Acids by Yarrowia lipolytica: Combining Molecular Design and TALEN to Engineer the Fatty Acid Synthase, ACS Synth. Biol. 2017, 6, 1870-1879.

In some embodiments, the microbial cell further comprises a biosynthetic pathway producing one or more lactones. In some embodiments, the microbial cell expresses one or more enzymes having fatty acid hydroxylase activity. In some embodiments, the enzyme is a cytochrome P450 enzyme (such as a CYP505 enzyme or derivative thereof). In certain embodiments, the enzymes described herein are engineered for hydroxylase activity at the desired position of a desired fatty acid substrate, to allow for production of the desired lactone. In some embodiments, the cell further expresses a cytochrome P450 reductase. In some embodiments, the P450 enzyme comprises a domain having fatty acid hydroxylase activity and CPR domain.

CYP505E3 from Aspergillus terreus (SEQ ID NO: 18) is a self-sufficient P450 enzyme comprising a reductase domain, and which can catalyze in-chain hydroxylation of alkanes, fatty alcohols, and fatty acids, including at the @-7 position. In some embodiments, the P450 enzyme comprises a domain having fatty acid hydroxylase activity, wherein the domain having fatty acid hydroxylase activity comprises an amino acid sequence that has at least 80%, or at least 90%, or at least 95%, or at least 97%, or at least 98%, or 100% sequence identity to amino acids 1 to 461 of SEQ ID NO: 18. In some embodiments, the P450 enzyme also comprises a CPR domain, which is optionally the CPR domain of SEQ ID NO: 18 or a derivative thereof (i.e., comprising an amino acid sequence that is at least about 70%, or at least about 80%, or at least about 90%, or at least about 95%, or at least about 97% identical thereto), or optionally a heterologous CPR domain.

In some embodiments, the enzyme hydroxylates 8:0, 10:0, and 12:0 fatty acids, thereby forming one or more lactones selected from δ-dodecalactone, δ-decalactone, γ-dodecalactone, γ-decalactone, and δ-octalactone. In some embodiments, the microbial cell expresses a biosynthetic pathway that produces two or more lactones selected from δ-dodecalactone, δ-decalactone, γ-dodecalactone, γ-decalactone, and δ-octalactone, upon contacting the cells with the corresponding fatty acid substrate (as described herein). In some embodiments, the microbial cell expresses a biosynthetic pathway producing δ-dodecalactone, δ-decalactone, γ-dodecalactone, γ-decalactone, and δ-octalactone, upon contacting the cells with the corresponding fatty acid substrate (as described herein).

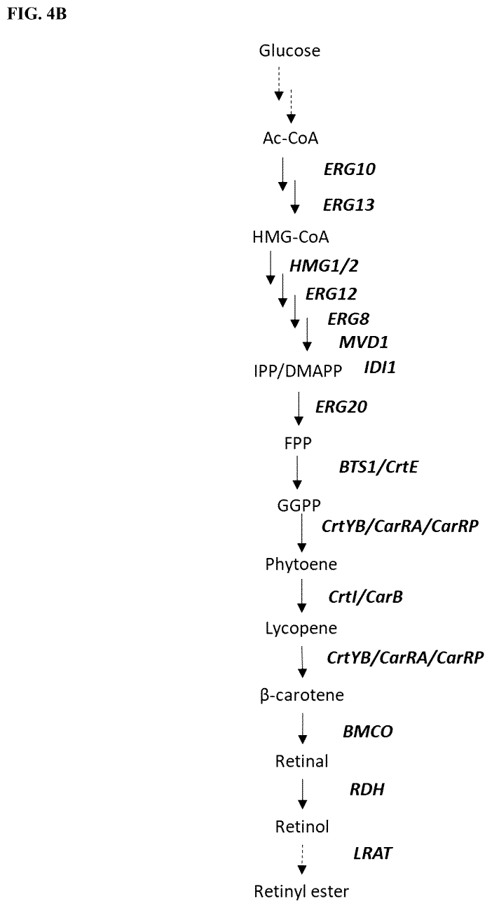

In some embodiments, the cell further produces β-carotene through a heterologous biosynthetic pathway, which can provide desirable color attributes for products such as butter and others. For example, the microbial cell can express heterologous biosynthetic enzymes such as a phytoene desaturase and/or a bifunctional lycopene cyclase/phytoene synthase. In some embodiments, the cell further produces vitamin A (e.g., from β-carotene substrate). In some embodiments, the microbial cell expresses a β-carotene 15,15′-monooxygenase, a retinal dehydrogenase and/or a lecithin: retinol acyltransferase. Precursors for β-carotene and vitamin A will be supplied by the mevalonic acid pathway (MVA) or non-mevalonic acid pathway (MEP pathway), which can be complemented or engineered for improved productivity. The MVA and MEP pathways produce iso-pentenyl pyrophosphate (IPP) and dimethylallyl pyrophosphate (DMAPP) precursors, which can be converted to farnesyl diphosphate. The MVA pathway is illustrated in FIG. 4B.

In some embodiments, the microbial host cell is engineered to express or overexpress one or more enzymes in the MEP and/or MVA pathways to catalyze IPP and DMAPP biosynthesis from sugars such as glucose or other carbon source.

In some embodiments, the microbial host cell is engineered to express or overexpress one or more enzymes of the MVA pathway. The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-COA) (e.g., by action of HMG-COA synthase (HMGS)); (c) converting HMG-CoA to mevalonate (e.g., by action of HMG-COA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the microbial host cell expresses or overexpresses one or more of acetoacetyl-CoA thiolase, HMGS, HMGR, MK, PMK, and MPD or modified variants thereof, which results in the increased production of IPP and DMAPP. In some embodiments, FPP is produced at least in part by metabolic flux through an MVA pathway, and wherein the microbial host cell has at least one additional gene copy of one or more of acetoacetyl-CoA thiolase, HMGS, HMGR, MK, PMK, MPD, or modified variants thereof.

The MEP pathway (endogenous to bacterial hosts), and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the microbial host cell expresses or overexpresses of one or more of dxs, ispC, ispD, ispE, ispF, ispG, ispH, idi, ispA, or modified variants thereof, which results in the increased production of IPP and DMAPP. In some embodiments, FPP is produced at least in part by metabolic flux through an MEP pathway, and wherein the microbial host cell has at least one additional gene copy of one or more of dxs, ispC, ispD, ispE, ispF, ispG, ispH, idi, ispA, or modified variants thereof.

In some embodiments, the MEP pathway of the microbial host cell is engineered to increase production of IPP and DMAPP from glucose as described in U.S. Pat. Nos. 10,662,442 and/or 10,480,015, the contents of which are hereby incorporated by reference in their entireties. For example, in some embodiments the microbial host cell overexpresses MEP pathway enzymes, with balanced expression to push/pull carbon flux to IPP and DMAPP. In some embodiments, the microbial host cell is engineered to increase the availability or activity of Fe-S cluster proteins, so as to support higher activity of IspG and IspH, which are Fe-S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux.

In alternative embodiments, the microbial host cell is not engineered to increase production of FPP from MEP or MVA pathway precursors, but FPP or precursor compound is fed to the cells to provide FPP substrate for-carotene or Vitamin A production. In still other embodiments, microbial cells express an isoprenol utilization pathway as described in U.S. Pat. No. 11,034,980, which is hereby incorporated by reference in its entirety. Such cells can produce IPP and DMAPP precursors from prenol and/or isoprenol substrate provided to the culture.

In certain embodiments, fatty acids and/or fatty acid esters are fed to the cells to impact the fatty acid composition of triacylglycerides, including the composition of short and/or medium chain fatty acids, as well as saturated fatty acid composition, of the triacylglycerides. This allows the microbial cell to approach the desired fatty acid profile. For example, in some embodiments, the microbial cell is fed one or a combination of C4:0, C6:0, C8:0, C10:0, C12:0, C14:0, and C16:0.

As discussed in more details below, the microbial cell may be a yeast or fungal cell, or in some embodiments, a bacterial cell. Accordingly, where genes are disrupted or inactivated according to embodiments of this disclosure, the disputed or inactivated gene will depend on the species of the host. For ease of understanding, unless stated otherwise, the gene names in this disclosure are Yarrowia lipolytica genes. A person of ordinary skill will understand how to identify homologs, orthologs or paralogs for a different host species. Further, strains may be engineered for increased expression of certain genes (such as by gene complementation or editing of expression control sequences) or for decreased activity of certain genes (such as through loss-of-function mutation(s) or editing of expression control sequences), and such derivatives may generally comprise an amino acid sequence that is at least 70%, or at least 80%, or at least 90%, or at least 95%, or at least 98% identical to the reference amino acid sequence.

The similarity or identity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215:403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

Expression of enzymes can be tuned for optimal activity, using, for example, gene modules (e.g., operons) or independent expression of the enzymes. For example, expression of the genes can be regulated through selection of promoters, such as inducible or constitutive promoters, with different strengths (e.g., strong, intermediate, or weak). Additionally, expression of genes can be regulated through manipulation of the copy number of the gene in the cell. In some embodiments, expression of genes can be regulated through manipulating the order of the genes within a module, where the genes transcribed first in an operon are generally expressed at a higher level. In some embodiments, expression of genes is regulated through integration of one or more genes into the chromosome.

Optimization of expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced with heterologous genes by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

In accordance with this disclosure, where genes are deleted, genes can be deleted in whole or in part (i.e., inactivated), which can include deletion of coding sequences and/or expression control sequences.

In some embodiments, the microbial cell is a yeast or fungal cell. In some embodiments, the yeast or fungal cell belongs to a genus selected from Aspergillus, Aurantiochytrium, Bastobotyrs, Candida, Claviceps, Cryptococcus, Cunninghamella, Geotrichum, Hansemula, Issatchenkia, Kluyveromyces, Kodamaea, Leucosporidiella, Linderna, Lipomyces, Mortierella, Myxozyma, Mucor, Occultifur, Ogataea, Penicillium, Phaffia, Pichia, Prototheca, Rhizopus, Rhodosporidium, Rhodotorula, Saccharomyces, Scheffersomyces, Schizosaccharomyces, Sporidiobolus, Sporobolomyces, Starmerella, Tremella, Trichosporon, Wickerhamomyces, Waltomyces, and Yarrowia. In some embodiments, the yeast or fungal cell belongs to a species selected from Yarrowia lipolytica, Yarrowia phangngensis, Pichia kudriavzevii, Saccharomyces cerevisiae, Pichia pastoris, Kluyveromyces marxianus, Rhodosporidium toruloides, Sporidiobolus ruinenii, Sporidiobolus salmonicolor, Aspergillus oryzae, Mortierella isabellina, Waltomyces lipofer, Candida tropicalis, Candida boidinii, Scheffersomyces stipitis, Mucor circinelloides, Ashbya gossypii, Trichoderma harzianium, Pichia guilliermondii, Kodamaea ohmeri, Rhodotorula aurantiaca, Lindnera saturnus, Penicillium roqueforti, Lipomyces starkeyi, and Bastobotyrs adeninivorans. In some embodiments, the yeast or fungal cell is Yarrowia lipolytica. In some embodiments, the yeast or fungal cell is Yarrowia phangngensis.

In some embodiments, the microbial cell is a bacterial cell. In some embodiments, the microbial cell is a bacterium that accumulates significant quantities of triacylglycerols. In some embodiments, the bacterial cell belongs to a genus selected from Acidovorax, Acinetobacter, Actinomyces, Alcanivorax, Arthrobacter, Brevibacterium, Bacillus, Clostridium, Corynebacterium, Dietzia, Escherichia, Gordonia, Marinobacter, Mycobacterium, Micrococcus, Micromonospora, Moraxella, Nocardia, Pseudomonas, Psychrobacter, Rhodococcus, Salmonella, Streptomyces, Thalassolituus, and Thermomonospora. In some embodiments, the bacterial cell belongs to a species selected from Rhodococcus opacus, Acinetobacter calcoaceticus, Streptomyces coelicolor, Rhodococcus jostii, and Acinetobacter baylyi.

In one aspect, the present disclosure relates to a method for making a composition comprising milk fat triglycerides. In some embodiments, the method comprises culturing a microbial cell according to any of the embodiments disclosed herein in the presence of fatty acid substrates. In some embodiments, the fatty acid substrates comprise C4:0, C6:0, C8:0, C10:0, C12:0, and C14:0 fatty acid substrates. The composition comprising milk fat triglycerides can be recovered from the culture. In some embodiments, the fatty acid substrates are added to the culture, optionally as alkyl esters or glycerides. In some embodiments, the fatty acid substrates are synthesized by the cell.

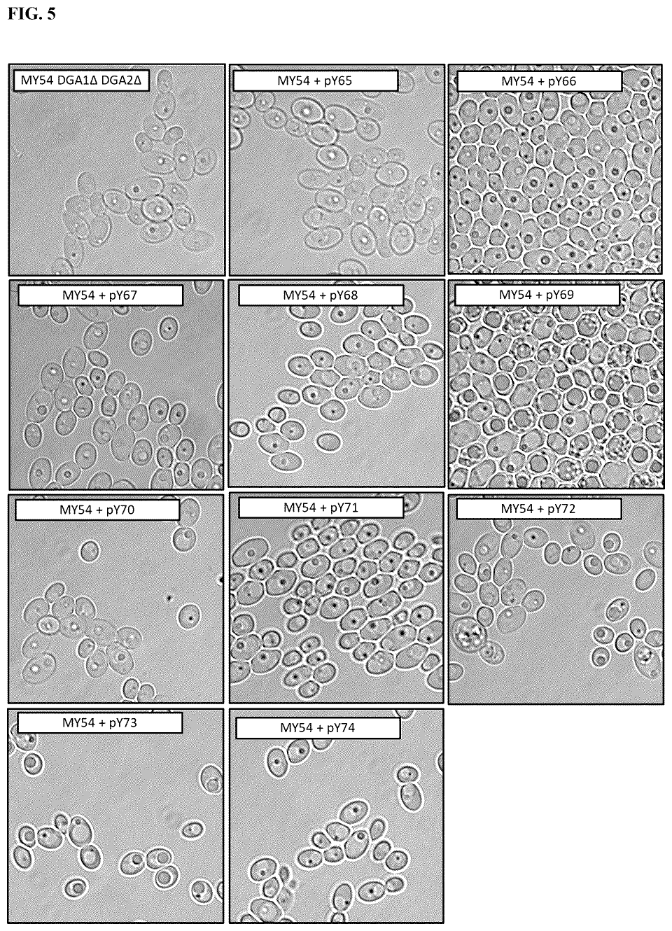

In some embodiments, the composition is recovered by separating the wet cell mass, and purifying the compositions from the wet cell mass. In some embodiments, the cells are mechanically or enzymatically disrupted. In some embodiments, the composition is extracted using an organic solvent. Exemplary recovery processes are shown in FIGS. 5A-D.

The host cells and methods are further suitable for commercial production of the composition, that is, the cells and methods can be productive at commercial scale. In some embodiments, the size of the culture is at least about 100 L, or at least about 200 L, or at least about 500 L, or at least about 1,000 L, or at least about 10,000 L, or at least about 50,000 L, or at least about 100,000 L, or at least about 200,000 L, or at least about 500,000 L, or at least about 1,000,000 L. In various embodiments, the culturing is conducted in batch culture.

In another aspect, the present disclosure provides a composition or product (e.g., a beverage or food product) comprising milk fat triglycerides made according to the present disclosure.

In another aspect, the disclosure provides methods for making a product comprising milk fat triglycerides. The method comprises incorporating the composition comprising milk fat triglycerides of the instant disclosure into a product (i.e., a food, beverage, flavor, or food additive product). Examples of products in which the compositions may be used include, but are not limited to, food products, beverages, flavors, and food additives. Exemplary products include milk, cheese, butter, yogurt, frozen yogurt, gelato, milk chocolate, cream (e.g., heavy cream, light cream, sour cream, etc.), ice cream, cream cheese, custard, anhydrous milk fat, condensed milk, milk powder, and evaporated milk.

Unless defined otherwise, all technical and scientific terms used herein have the same meaning as commonly understood by one of ordinary skill in the art to which the invention pertains.

As used herein, the term “about” means+10% of an associated numerical value, unless the context requires otherwise.

### EXAMPLES

A biosynthetic scheme for metabolic engineering of microorganisms to produce components of milk fat is shown in FIG. 4. The following Examples illustrate some non-limiting embodiments towards the metabolic engineering and purification of the fat produced by the microorganisms.

### Example 1. Engineering C4-C12 Fatty Acid Acyl-CoA Synthetase Activity

Yarrowia lipolytica converts free fatty acids into acyl-CoAs via two endogenous acyl-CoA synthetases, FAA1 and FAT1. FAA1 is localized to the cytosol and is involved in fatty acid incorporation into larger biomolecules. FAT1 is localized to the peroxisomes and is involved in fatty acid degradation for carbon and energy. Both enzymes favor long chain fatty acids, with little activity with short and medium chain fatty acids.

To produce triglycerides with short chains at the sn-3 position, supply of short and medium chain acyl-CoAs in the cytosol are required. Therefore, heterologous acyl-CoA synthetases were expressed and screened for their activity with short and medium chain (C4-C12) fatty acid substrates.

Yarrowia lipolytica MY16 was transformed with plasmids carrying acyl-CoA synthetases (SEQ ID NOs: 1 to 7), and cell free extracts were prepared. Acyl-CoA synthetase (ACS) activity was quantified by an in vitro assay that measured the conversion of CoA and carboxylic acid to acyl-CoA via quantification of the residual free CoA concentration after incubation with cell free extract. The results of the assay are shown in Table 1. From this in vitro assay, the enzyme of SEQ ID NO: 2 was found to have activity >20 μmol min⁻¹ mg protein⁻¹ with C4, C5, C6, and C7 fatty acids when expressed in Y. lipolytica. The enzyme of SEQ ID NO: 3 was found to have activity >20 μmol min⁻¹ mg protein⁻¹ with C4, C5, C6, C7, C8, C9, C10, C11, and C12 fatty acids, while the enzyme of SEQ ID NO: 6 had activity with C5, C6, C7, C8, C9, C10, and C11 when expressed in Y. lipolytica MY16.

### Example 2. Expression of PpLvaE and RpDcaA Acyl-CoA Synthetases in a DGA1Δ DGA24 Yarrowia lipolytica Strain

The Yarrowia lipolytica diglyceride acyltransferases DGA1 (YALI0E3269g) and DGA2 (YALIOD07986g) were targeted for gene deletion. These native DGA enzymes are responsible for the majority of triacylglycerol synthesis, but do not accept short chain acyl-CoA substrates. A DGA1Δ DGA24 Yarrowia lipolytica strain background may be suitable for expression of heterologous diglyceride acyltransferases with the ability to incorporate short- and medium-chain acyl-CoA substrates into the sn-3 position of triglycerides. DGA1 and DGA2 were deleted by homologous recombination in Y. lipolytica MY16, leading to strain MY43 containing DGA1Δ DGA2Δ and a hygromycin selection marker. The hygromycin marker was subsequently removed resulting in a DGA1Δ DGA2Δ strain designated MY54.

A vector co-expressing the Acyl-CoA Synthetase of SEQ ID NO: 2 from the Y. phangngensis TEF1 promoter and the Acyl-CoA Synthetase of SEQ ID NO: 3 from the Y. lipolytica TEF1 promoter was created using standard molecular biology techniques and designated pMY64, and transformed into MY43. Colonies were assayed for in vitro short and medium chain acyl-CoA synthetase activity via the method described in Example 1, and found to have an average of 35 μmol min⁻¹ mg protein⁻¹ activity with butyric acid and 14 μmol min⁻¹ mg protein⁻¹ activity with octanoic acid substrates.

### Example 3. Expression of Heterologous DGAT Enzymes in Strain MY54

DNA sequences (codon optimized for expression in Y. lipolytica) were created for the enzymes BtDGAT1, ChDGAT1, OaDGAT1, BbDGAT1, BtDGAT2, OaDGAT2, BtDGAT2L6, ChDGAT2L6, CaDGAT1, and EaDAcT (SEQ ID NOs: 8-17) and assembled in a gene cassette driven by the Y. lipolytica TEF1 promoter in a Y. lipolytica expression vector. These vectors were given the designations pY65 (BtDGAT1), pY66 (ChDGAT1), pY67 (OaDGAT1), pY68 (BbDGAT1), pY69 (BtDGAT2), pY70 (OaDGAT2), pY71 (BtDGAT2L6), pY72 (ChDGAT2L6), pY73 (CaDGAT1), and pY74 (EaDAcT) and transformed into strain MY54. Isolated strains were cultured in minimal glucose medium for 95 hours at 30° C. and 900 rpm and observed by microscopy for the presence of increased lipid body size. Images of these strains are shown in FIG. 5.

### Example 4: Yarrowia lipolytica Strain Accumulating Lipid with 6:0 Fatty Acid Acyl Chains

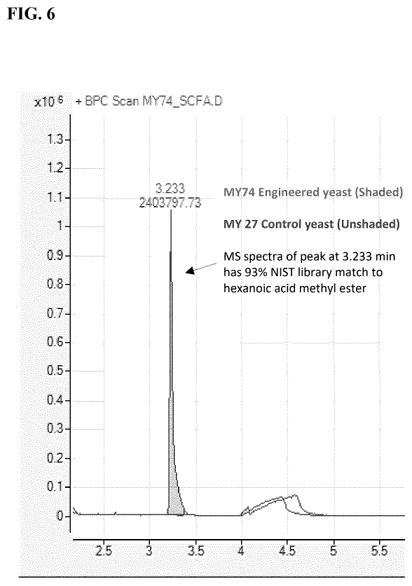

Yarrowia lipolytica strain MY54 with genotype DGA1Δ DGA2Δ was transformed with a non-targeted integration cassette containing the Bos taurus DGAT2 enzyme expressed from the Y. lipolytica TEF1 promoter region. Several colonies were re-isolated to single colonies and incubated in defined minimal glucose medium for 72 hours at 30° C. at 250 rpm. Cell cultures were then examined by microscopy for the presence of lipid bodies, and one culture with visibly large lipid bodies was designed strain MY63. MY63 was subsequently transformed with plasmid pY64 and a transformant was isolated and designated MY74. MY74 and an unmodified control strain MY27 were cultivated in minimal medium containing glycerol and 4.8 mM hexanoic acid. After 94 hours of incubation cells were harvested and lipid was extracted from both samples via a modified Bligh and Dwyer method. Lipids were dissolved in methyl-tert-butyl ether (MBTE) at a concentration of 100 mg/mL and subjected to transesterification with a 100-fold volume excess of 0.5 N sodium methoxide and incubated at 50° C. for 30 minutes. A reference sample of ghee butter was included for analysis. Fatty acid methyl esters derived from yeast or ghee butter lipid were analyzed by gas chromatography-mass spectrometry. Compounds were identified via comparison to the NIST mass spectra database and via a Supelco 37 FAME reference standard. The chromatogram of the MY74 lipid derived sample contained a peak with the same retention time as hexanoic acid methyl ester and a 93% match to the NIST mass spec reference library. FIG. 6. This peak was also present in ghee butter but absent in the lipid sample from MY27.

### Example 5. Altering Triacylglyceride Profile by Feeding Short, Medium, and Saturated Fatty Acids to an Engineered Yeast

The engineered Yarrowia lipolytica yeast strain MY 167 was created by transforming vector pY111 into the DGA1Δ DGA2Δ parental strain MY54. Vector pY111 contains a multi-gene expression cassette containing BtDGAT2, PplvaE, and RpDcaA expressed via separate constitutive promoters. To assay triacylglyceride composition, cells were cultivated in a 96 well plate shaken at 30° C. with a glucose minimal medium. At 24 hours post-inoculation fatty acids, either provided as free fatty acids (C6-C10) or ethyl esters (C12-C16), were directly added to the fermentation medium. The cells were continually shaken for 70 hours post-inoculation and then the cell culture was harvested, washed, dried, and subjected to methanolic chloride catalyzed transesterification and analyzed by gas chromatography with a flame ionization detector to determine lipid acyl-chain composition. Results of the lipid profile analysis are shown in Table 2. The results demonstrate that the short, medium, and saturated fatty acid composition of the triacylglycerides can be altered by feeding fatty acids of similar species to the engineered yeast strain. This allows the engineered strain to approach the fatty acid profile of a representative bovine milk fat. For example, addition of C14:0 fatty acid to the fermentation medium increases the C14:0 triacylglyceride content from 0.9% to 32.7%. The bovine milk fat C14:0 target is 12.0%.

### Example 6. Altering Triacylglyceride Profile by Inactivation of FAD2 Delta-12 Desaturase and ELO1 Fatty Acid Elongase

Yarrowia lipolytica yeast strain MY167 was transformed with a gene disruption cassette targeting the FAD2 (YALIOB10153g) locus. The resulting strain, MY168, was cultivated in a 96 well plate shaken at 30° C. with a glucose minimal medium for 140 hours. The cell culture was then harvested, washed, dried, and subjected to methanolic chloride catalyzed transesterification and analyzed by gas chromatography with a flame ionization detector to determine lipid acyl-chain composition. Results of the lipid profile analysis are shown in Table 3. Results demonstrate that the polyunsaturated fatty acid content can be reduced from 6.4% to 0.0% by deletion of the FAD2 gene. A representative bovine milk fat polyunsaturated fat content is 2%.

Yarrowia lipolytica yeast strain MY168 was transformed with a gene disruption cassette targeting the ELO1 (YALI0F06754g) locus. The resulting strain, MY174, was cultivated in a 96 well plate shaken at 30° C. with a glucose minimal medium for 140 hours. The cell culture was then harvested, washed, dried, and subjected to methanolic chloride catalyzed transesterification and analyzed by gas chromatography with a flame ionization detector to determine lipid acyl-chain composition. Results of the lipid profile analysis are shown in Table 3. Results demonstrate that the C16 to C18 fatty acid species ratio can be engineered from 0.33 (Yeast MY168) to 1.01 (Yeast MY14) via deletion of the ELO1 gene. A representative bovine milk fat C16 to C18 fatty acid species ratio is 1.58.

### Example 7. Purification of Milk Fat Triglyceride from Yarrowia lipolytica

The milk fat triglyceride will be produced intracellularly and accumulate inside specialized organelles called lipid bodies. To separate the milk fat product from whole yeast cells, the following downstream separation processes can be used.

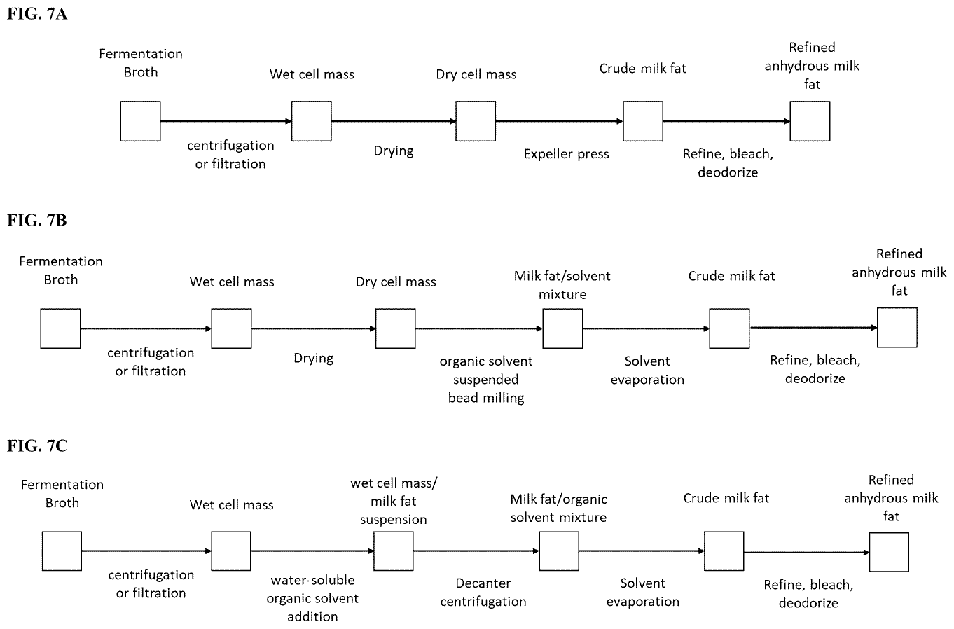

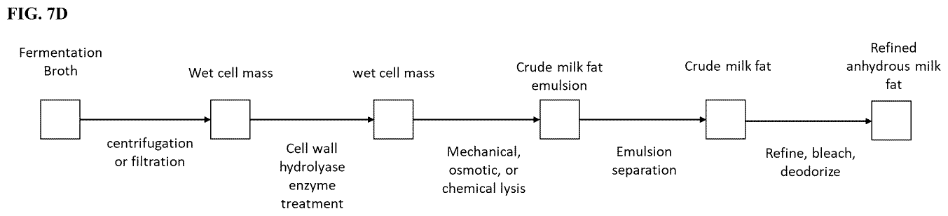

As shown in FIG. 7A, cell mass can be separated from the fermentation broth via centrifugation or filtration. The cell mass will be dried and pressed via an expeller press to create a crude milk fat lipid and a cell pressate. Crude milk fat can be further processed via refining, bleaching, and de-odorizing to produce a refined milk fat.

As shown in FIG. 7B, dry cell mass can be suspended in an organic solvent (e.g., hexane, chloroform, methanol, cyclopentyl methyl ether, isooctane, ethyl acetate, ethanol, acetone, isopropyl alcohol, or a mixture thereof) and bead milled to release intracellular lipids to the solvent phase. Milk fat laden solvent will then be separated from the residual solids and solvent evaporated to produce a crude milk fat. The crude milk fat can be further processed to a refined milk fat product. This process is feasible for a wide range of lipid contents, from 30% to >70% w/w of the total cell mass. Variations of this process can replace bead milling with high pressure homogenization (French press), sonication, microwave, pulsed electric field, or other mechanical cell disruption methods.

As shown in FIG. 7C, a wet cell mass can be used for an aqueous phase extraction with addition of a water miscible organic solvent, such as ethanol. The water-solvent-oil-cell mass mixture will be mixed in process conditions that encourage cell lysis, and then oil will be separated into an organic phase via decanter centrifugation. The addition of organic solvent inhibits the formation of a stable oil-water emulsion. See, U.S. Pat. No. 5,928,696, the entire contents of which are hereby incorporated by reference.

As shown in FIG. 7D, cellular lysis may be improved by the addition of cell wall degrading enzymes. Yeast and fungal cell walls are composed of the polysaccharide chitin as well as proteins. Cell wall degrading enzymes such as endo-chitinases, chitobiosidases, lytic polysaccharide monooxygenases, N-acetylglucosaminidases, and proteases weaken and remove the yeast cell wall, facilitating subsequent mechanical, chemical, or osmotic separation steps.

SEQUENCES
Acyl-CoA synthetase Enzymes
SEQ ID NO: 1
Escherichia coli FadK (ecFadK)
MHPTGPHLGPDVLFRESNMKVTLTFNEQRRAAYRQQGLWGDASLADYWQQTARAMPDKIA
VVDNHGASYTYSALDHAASCLANWMLAKGIESGDRIAFQLPGWCEFTVIYLACLKIGAVS
VPLLPSWREAELVWVLNKCQAKMFFAPTLFKQTRPVDLILPLQNQLPQLQQIVGVDKLAP
ATSSLSLSQIIADNTSLTTAITTHGDELAAVLFTSGTEGLPKGVMLTHNNILASERAYCA
RLNLTWQDVFMMPAPLGHATGFLHGVTAPFLIGARSVLLDIFTPDACLALLEQQRCTCML
GATPFVYDLLNVLEKQPADLSALRFFLCGGTTIPKKVARECQQRGIKLLSVYGSTESSPH
AVVNLDDPLSRFMHTDGYAAAGVEIKVVDDARKTLPPGCEGEEASRGPNVFMGYFDEPEL
TARALDEEGWYYSGDLCRMDEAGYIKITGRKKDIIVRGGENISSREVEDILLQHPKIHDA
CVVAMSDERLGERSCAYVVLKAPHHSLSLEEVVAFFSRKRVAKYKYPEHIVVIEKLPRTT
SGKIQKFLLRKDIMRRLTQDVCEEIE
SEQ ID NO: 2
Pseudomonas putida LvaE (ppLvaE)
MMVPTLEHELAPNEANHVPLSPLSFLKRAAQVYPQRDAVIYGARRYSYRQLHERSRALAS
ALERVGVQPGERVAILAPNIPEMLEAHYGVPGAGAVLVCINIRLEGRSIAFILRHCAAKV
LICDREFGAVANQALAMLDAPPLLVGIDDDQAERADLAHDLDYEAFLAQGDPARPLSAPQ
NEWQSIAINYTSGTTGDPKGVVLHHRGAYLNACAGALIFQLGPRSVYLWTLPMFHCNGWS
HTWAVTLSGGTHVCLRKVQPDAINAAIAEHAVTHLSAAPVVMSMLIHAEHASAPPVPVSV
ITGGAAPPSAVIAAMEARGFNITHAYGMTESYGPSTLCLWQPGVDELPLEARAQFMSRQG
VAHPLLEEATVLDTDTGRPVPADGLTLGELVVRGNTVMKGYLHNPEATRAALANGWLHTG
DLAVLHLDGYVEIKDRAKDIIISGGENISSLEIEEVLYQHPEVVEAAVVARPDSRWGETP
HAFVTLRADALASGDDLVRWCRERLAHFKAPRHVSLVDLPKTATGKIQKFVLREWARQQE
AQIADAEH
SEQ ID NO: 3
Rhodopseudomonas palustris DcaA (rpDcaA)
MSFAYFDWIAHHAEVRPERIAVVDLASSRKISYRAMDERVDRLAAHLAALGVGRGDRVAV
LALNAVETLEVQFACFRLGAIFVPLNVRLTVHELSYIVGDAAPRVLAHDDELAPMAKELK
AACSVPHLLAFGAAYEAALAASPRLGASEPVTLDDVSTIMYTSGTTGKPKGAMITHGMTF
INAVNLGIPAFISQRTVFLCVLPLFHTGGLNCYTNPVLHAGGTTLLMRAFDPGAALSIIG
DPSVGLTHFFGVPSIYQFMCQHPAFAATDLSRLQIAGVGGAPMPVPLLKIWQERGCALVQ
GYGMTETSPAVLMLDADDAARKAGSAGKPVLHADLKIVGPDGDPVKPGEMGELWVKGPNI
TPGYWNRPDANRTSFTDGWLHTGDAARVDDEGFYYIVDRTKDMYISGGENVYPAEVEDVL
YQLPEIAEAAVIGAPDPQWGETGVAVVALKPGQELSEAKLLAHCRERLARFKCPQRVSFV
EALPRNATGKVHKPTLRERILVRETADA
SEQ ID NO: 4
Rhodopseudomonas palustris DcaC (rpDcaC)
MTSLEATGGVPGPGRIGRVAIGDILRKSARRFPDRVALTDGGRSVTYTELERDANRFANA
LVARGLKPGAKISTVCNNSIEFVKALFGIHRAGLVWVPINTMLGPDDMGYILDHAGVKVA
VIDDNLHGQPERRAALEARGIDLIAINLAGKAADTGLPVFDQLIEGLSEIEPDVAFDDRD
LAMIIYTSGTTSRPKGAMHCHLAVTMAVMSNAIEMQLSRKDGITGQFPLFHCAAHVLLLS
YLIVGGQMAIMRGFDPVACMEAIQRNKLTVFIGLPLMYQVILDHPRRKEFDLSSLRCCIY
TMAPMPRPLLERAIAELCPTFVQPSGQTEMYPATTMSQPDRQLARFGNYWGESTLVNETA
IMDDAGNLLPPGEVGEIVHRGPNVMLGYYKDPEATEAARKFGWHHTGDLALIDEHGEVLF
LDRKKDMIKSGGENVASIKIEETLLAHPSVMNAAVVGLPHPQWGEAVSGFVKLKPGASAT
EAEIVEHCKKHLGGFQVPKLLRIVDEMPMTATGKLRKVELRNQFTDHFMLGQTG
SEQ ID NO: 5
Cannabis sativa AAE1 (csAAE1)
MGKNYKSLDSVVASDFIALGITSEVAETLHGRLAEIVCNYGAATPQTWINIANHILSPDL
PFSLHQMLFYGCYKDFGPAPPAWIPDPEKVKSTNMGALLEKRGKEFLGVKYKDPISSFSH
FQEFSVRNPEVYWRTVLMDEMKISFSKDPECILRRDDDINNPGGSEWLPGGYLNSAKNCL
NVNSNKKLNDTMIVWRDEGNDDLPLNKLTLDQLRKRVWLVGYALEEMGLEKGCAIAIDMP
MHVDAVVIYLAIVLAGYVVVSIADSFSAPEISTRLRLSKAKAIFTQDHIIRGKKRIPLYS
RVVEAKSPMAIVIPCSGSNIGAELRDGDISWDYFLERAKEFKNCEFTAREQPVDAYTNIL
FSSGTTGEPKAIPWTQATPLKAAADGWSHLDIRKGDVIVWPTNLGWMMGPWLVYASLLNG
ASIALYNGSPLVSGFAKFVQDAKVTMLGVVPSIVRSWKSTNCVSGYDWSTIRCFSSSGEA
SNVDEYLWLMGRANYKPVIEMCGGTEIGGAFSAGSFLQAQSLSSFSSQCMGCTLYILDKN
GYPMPKNKPGIGELALGPVMFGASKTLLNGNHHDVYFKGMPTLNGEVLRRHGDIFELTSN
GYYHAHGRADDTMNIGGIKISSIEIERVCNEVDDRVFETTAIGVPPLGGGPEQLVIFFVL
KDSNDTTIDLNQLRLSFNLGLQKKLNPLFKVTRVVPLSSLPRTATNKIMRRVLRQQFSHF
E
SEQ ID NO: 6
Arabidopsis thaliana ACS (atACS)
MASEENDLVFPSKEFSGQALVSSPQQYMEMHKRSMDDPAAFWSDIASEFYWKQKWGDQVF
SENLDVRKGPISIEWFKGGITNICYNCLDKNVEAGLGDKTAIHWEGNELGVDASLTYSEL
LQRVCQLANYLKDNGVKKGDAVVIYLPMLMELPIAMLACARIGAVHSVVFAGFSADSLAQ
RIVDCKPNVILTCNAVKRGPKTINLKAIVDAALDQSSKDGVSVGICLTYDNSLATTRENT
KWQNGRDVWWQDVISQYPTSCEVEWVDAEDPLFLLYTSGSTGKPKGVLHTTGGYMIYTAT
TFKYAFDYKSTDVYWCTADCGWIGGHSYVTYGPMLNGATVVVFEGAPNYPDPGRCWDIVD
KYKVSIFYTAPTLVRSLMRDDDKFVTRHSRKSLRVLGSAGEPINPSAWRWFFNVVGDSRC
PISDTWGQTETGGFMITPLPGAWPQKPGSATFPFFGVQPVIVDEKGNEIEGECSGYLCVK
GSWPGAFRTLFGDHERYETTYFKPFAGYYFSGDGCSRDKDGYYWLTGRVDDVINVSGHRI
GTAEVESALVLHPQCAEAAVVGIEHEVKGQGIYAFVTLLEGVPYSEELRKSLVLMVRNQI
GAFAAPDRIHWAPGLPKTRSGKIMRRILRKIASRQLEELGDTSTLADPSVVDQLIALADV
SEQ ID NO: 7
Saccharomyces cerevisiae FAA2 (scFAA2)
MAAPDYALTDLIESDPRFESLKTRLAGYTKGSDEYIEELYSQLPLTSYPRYKTFLKKQAV
AISNPDNEAGFSSIYRSSLSSENLVSCVDKNLRTAYDHFMFSARRWPQRDCLGSRPIDKA
TGTWEETFRFESYSTVSKRCHNIGSGILSLVNTKRKRPLEANDFVVAILSHNNPEWILTD
LACQAYSLTNTALYETLGPNTSEYILNLTEAPILIFAKSNMYHVLKMVPDMKFVNTLVCM
DELTHDELRMLNESLLPVKCNSLNEKITFFSLEQVEQVGCFNKIPAIPPTPDSLYTISFT
SGTTGLPKGVEMSHRNIASGIAFAFSTFRIPPDKRNQQLYDMCFLPLAHIFERMVIAYDL
AIGFGIGFLHKPDPTVLVEDLKILKPYAVALVPRILTRFEAGIKNALDKSTVQRNVANTI
LDSKSARFTARGGPDKSIMNFLVYHRVLIDKIRDSLGLSNNSFIITGSAPISKDTLLFLR
SALDIGIRQGYGLTETFAGVCLSEPFEKDVGSCGAIGISAECRLKSVPEMGYHADKDLKG
ELQIRGPQVFERYFKNPNETSKAVDQDGWFSTGDVAFIDGKGRISVIDRVKNFFKLAHGE
YIAPEKIENIYLSSCPYITQIFVFGDPLKTFLVGIVGVDVDAAQPILAAKHPEVKTWTKE
VLVENLNRNKKLRKEFLNKINKCTDGLQGFEKLINIKVGLEPLTLEDDVVTPTFKIKRAK
ASKFFKDTLDQLYAEGSLVKTEKL
Diacylglycerol O-acyltransferase Enzymes
SEQ ID NO: 8
Bos taurus (cow) DGAT1 (BtDGAT1)
MGDRGGAGGSRRRRTGSRPSIQGGSGPAAAEEEVRDVGAGGDAPVRDTDKDGDVDVGSGH
WDLRCHRLQDSLFSSDSGFSNYRGILNWCVVMLILSNARLFLENLIKYGILVDPIQVVSL
FLKDPYSWPALCLVIVANIFAVAAFQVEKRLAVGALTEQAGLLLHGVNLATILCFPAAVA
FLLESITPVGSVLALMVYTILFLKLFSYRDVNLWCRERRAGAKAKAALAGKAANGGAAQR
TVSYPDNLTYRDLYYFLFAPTLCYELNFPRSPRIRKRFLLRRLLEMLFLTQLQVGLIQQW
MVPAIQNSMKPFKDMDYSRIVERLLKLAVPNHLIWLIFFYWLFHSCLNAVAELMQFGDRE
FYRDWWNSESITYFWQNWNIPVHKWCIRHFYKPMLRRGSSKWAARTAVFLASAFFHEYLV
SIPLRMFRLWAFTGMMAQIPLAWIVGRFFRGNYGNAAVWLSLIIGQPVAVLMYVHDYYVL
NREAPAAGT
SEQ ID NO: 9
Capra hircus (goat) DGAT1 (ChDGAT1)
MGDRGGAGGSRRRRTGSRPSIQGGSRPAAAEEEVRDVGAGGDAPVRDTDKDGDVDVGSGH
WDLRCHRLQDSLFSSDSGFSNYRGILNWCVVMLILSNARLFLENLIKYGILVDPIQVVSL
FLKDPYSWPALCLVIVANIFAVAAFQVEKRLAVGALTEQAGLLLHGVNLATILCFPAAVA
FLLESITPVGSVLALMVYTILFLKLFSYRDVNLWCRERRAGAKAKAALAGKKANGGAAQR
TVSYPDNLTYRDLYYFLFAPTLCYELNFPRSPRIRKRFLLRRLLEMLFLTQLQVGLIQQW
MVPAIQNSMKPFKDMDYSRIVERLLKLAVPNHLIWLIFFYWLFHSCLNAVAELMQFGDRE
FYRDWWNSESITYFWQNWNIPVHKWCLRHFYKPMLRRGSSKWAARTGVFLASAFFHEYLV
SIPLRMFRLWAFTGMMAQIPLAWIVGRFFRGNYGNAAVWLSLIIGQPVAVLMYVHDYYVL
NREAPTAGT
SEQ ID NO: 10
Ovis aries (sheep) DGAT1 (OaDGAT1)
MGDRGGAGGSRRRRTGSRPSIQGGSRPAAAEEEVRDVGAGGDAPVRDTDKDGDVDVGSGH
WDLRCHRLQDSLFSSDSGFSNYRGILNWCVVMLILSNARLFLENLIKYGILVDPIQVVSL
FLKDPYSWPALCLVIVANIFAVAAFQVEKRLAVGALTEQAGLLLHGVNLATILCFPAAVA
FLLESITPVGSVLALMVYTILFLKLFSYRDVNLWCRERRAGAKAKAALAGKKANGGAAQR
TVSYPDNLTYRDLYYFLFAPTLCYELNFPRSPRIRKRFLLRRLLEMLFLTQLQVGLIQQW
MVPAIQNSMKPFKDMDYSRIVERLLKLAVPNHLIWLIFFYWLFHSCLNAVAELMQFGDRE
FYRDWWNSESITYFWQNWNIPVHKWCLRHFYKPMLRRGSSKWAARTGVFLASAFFHEYLV
SIPLRMFRLWAFTGMMAQIPLAWIVGRFFRGNYGNAAVWLSLIIGQPVAVLMYVHDYYVL
NREAPTAGT
SEQ ID NO: 11
Bubalus bubalis (domestic buffalo) DGAT1 (BbDGAT1)
MGDRGGAGGSRRRRTGSRPSIQGGSGPAAAEEEVRDVGAGGDAPVRDTDKDGDVDVGSGH
WDLRCHRLQDSLFSSDSGFSSYRGILNWCVVMLILSNARLFLENLIKYGILVDPIQVVSL
FLKDPYSWPALCLVIVANIFAVAAFQVEKRLAVGALTEQAGLLLHGVNLATILCFPAAVA
FLLESITPVGSVLALMVYTILFPKLFSYRDVNLWCRERRAGAKAKAALAGKKANGGAAQR
TVSYPDNLTYRDLYYFLFAPTLCYELNFPRSPRIRKRFLLRRLLEMLFLTQLQVGLIQQW
MVPAIQNSMKPFKDMDYSRIVERLLKLAVPNHLIWLIFFYWLFHSCPNAVAELMQFGDRE
FYRDWWNSESITYFWLNWNIPVHKWCIRHFYKPMLRRGSSKWAARTAVFLASAFFHGYLV
SIPLRMFRLWAFTGMMAQIPLAWIVGRFFRGNYGNAAVWLSLIIGQPVAVLMYVHDYYVL
NREAPAAGT
SEQ ID NO: 12
Bos taurus (cow) DGAT2 (BtDGAT2)
MKTLIAAYSGVLRGTGSSILSALQDLFSVTWLNRSKVEKQLQVISVLQWVLSFLVLGVAC
SVILMYTFCTDCWLIAVLYFTWLVFDWNTPKKGGRRSQWVRNWAVWRYFRDYFPIQLVKT
HNLLTSRNYIFGYHPHGIMGLGAFCNFSTEATEVSKKFPGIRPYLATLAGNFRMPVLREY
LMSGGICPVNRDTIDYLLSKNGSGNAIIIVVGGAAESLSSMPGKNAVTLRNRKGFVKLAL
RHGADLVPTYSFGENEVYKQVIFEEGSWGRWVQKKFQKYIGFAPCIFHGRGLFSSDTWGL
VPYSKPITTVVGEPITIPRLERPTQQDIDLYHAMYVQALVKLFDQHKTKFGLPETEVLEV
N
SEQ ID NO: 13
Ovis aries (sheep) DGAT2 (OaDGAT2)
MKTLIAAYSGVLRGTGSSILSALQDLFSVTWLNRSKVEKQLQVISVLQWVLSFLVLGVAC
SAILMYAFCTDCWLIAVLYFTWLVFDWNTPKKGGRRSQWVRNWAVWRYFRDYFPIQLVKT
HNLLTSRNYIFGYHPHGIMGLGAFCNFSTEATEVSKKFPGIRPYLATLAGNFRMPVLREY
LMSGGICPVNRDTIDYLLSKNGSGNAIIIVVGGAAESLSSMPGKNAVTLRNRKGFVKLAL
RHGADLVPIYSFGENEVYKQVIFEEGSWGRWVQKKFQKYIGFAPCIFHGRGLFSSDTWGL
VPYSKPITTVVGEPITIPKLEHPTQQDIDLYHAMYMEALVKLFDQHKTKFGLPETEVLEV
N
SEQ ID NO: 14
Bos taurus (cow) DGAT2L6 (BtDGAT2L6)
MAMAFLSQLNLQEILQTLSVLQWMPVYVFLGAIPIIVIPYFLVFTKFWMVSVLALAWLAY
DWNTHSQGGRRSAWVRNWTIWKYFQNYFPIKLVKTHDLSPRHNYIIASHPHGVLPYGTFI
NFATETTGFARIFPGITPYVATLEGIFWIPIVREYVMSMGVCPVSELALKYLLTQKGSGN
AVVIMVGGGAEALLCHPGATTVLLKQRKGFVKVALETGAYLVPSYSFGQNEVHNQETFPE
GTWKRFFQKALQDTLKKLLRLSVCTFHGRGLTRGSWGFLPFNHPITTVVGEPLPIPRIKK
PNEETVDKYHALYINALQKLFDEHKVQYGLSETQELTII
SEQ ID NO: 15
Capra hircus (goat) DGAT2L6 (ChDGAT2L6)
MAMAFLSQLNLQEILQTLSVLQWVPVYIFLGAIPIILIPYFLVFTKFWMVSVLALAWLAY
DWNTHSQGGRRSAWVRNWTIWKYFRNYFPVMLVKTHDLSPRQNYIIASHPHGILPYGIFI
NFATETTGFARIFPAITPYIATLEGIFWIPIVREYVMSMGVCPVSELALKYLLTQKGSGN
AVVIVVGGAAEALLCHPGASTVFLKQRKGFVKVALKTGAYLVPSYSFGQNEVHNQETFPE
GTWKRFFQKTLQNTFKKILGLNFCTFHGRGLIRGSWGFLPFNHPITTVVGEPLPIPRIKK
PNKETVDKYHALYINALRKLFDEHKVQYGLSETQELTIV
SEQ ID NO: 16
Cuphea avigera var pulcherrima DGAT1 (CaDGAT1)
MAAHRTSSPVHLKLAESPLSSRNIFKQNHEGLFNLCMVTLVAVIIRLFLENLLKYGWLMK
RDFWLSTFTAWPLFICSLGLPIFPLAAFVVEKLAQKNLLPEPIVLCSHVIITSASVLYPA
LVILRFDCALMSGIGLMLYSCALWLKLVSYAHTSYDMRCEAKSRLEGKSSADSKNGELPY
RVNIKDLAYFMVAPTLCYQLSYPRTQFIRKFWVARQVLKLILVNVVMGFIIEQYMIPVMH
NSKPPRRGYWLHFIERNLKLAVPSIGLWFCIFYSIFHLWLNIVAELLRFGDREFYKDWWN
AKNMEEYWKMWNIPVHRWMVRHLYGPCMKRKLPRWVAISISFLLSAVLHEICVSVPCHVF
QLWAFNGMMLQIPLVLSSKPLQKRFPSSKAGNVFFWFLFCIYGQPNCVLMYYHALMERRG
LRID
SEQ ID NO: 17
Euonymus alatus (Burning Bush) DACT (EaDACT)
MMDAHQEIKNFIKVWVQAMVCLSYAYYFSSRLPKGLLRLLSLLPVLYLLLIAPLNISSFI
LSSITGFFLAWLTTFKVISFAFDQGPLYPLPQNLLHFISIACLPITIKRNPSPKLKSTTN
PSPISHLLKKAFMSFPSKVLFHWVIAHLYQYKKYMDPNVVLVIYCCHVYVMLDISLSLCA
TLAEFLCGFDVEPQFKEPYLATSLQDFWGRRWNIIVSSVLRSTVYAPTRNIASYLIGSRW
AYFPAIIATFVVSGVMHDVVYYVYMMHMYPKWDMTGHFVLHGICEALEVEMKCKRSRSDK
WRRHPAVDWVMVMGFVMGTSVSLLFVPLLRDNVDQIVAEEYSILFNFVREKIVMLGTRFV
CGN
CYP505 Enzymes
SEQ ID NO: 18
Aspergillus terreus CYP505E3_1
MAIKETEQIPGPRPLPVVGNLFDMDLEHGLECLIRLADDFGPLFQITINGEKQIFATSQA
LVDELCDESRFHKAVMGGLEKLRMLASDGLFTAYHGERGWGIAHRILVPAFGPLRIRNMF
EEMNDVAQQLCLKWARQGSSTSINITDDFTRLTLDTIALCTMNFRLNSFYNNETMHPFVK
SMLYVLRESDIQSMLPGIANCIRVKARSRMSKHIQLMRNMARGIIQERRDQAEPVDDLLN
TLLNGRDPVTGEGMSDDLIINNVITFLIAGHETTSGLLSFTFYYLLQNPHILERAQNEVD
EVTGGERITVQHLGRLTYIDAILKESLRLMPTAPAFTVTPKKPEVLGGAWAIDAGQAVNV
LLPVCLRDRSVFGPDADEFRPERMLEENFSKLPPNSWKPFGNGERSCIGRAFAWQEAQLV
VAMVLQTFDLVPDDPSYKLRIKETLTIKPDGFRVRATLRRGQSATGLSQGSMSASGATSS
VASPGPPAATGAQSNPAGGQRISFFYGSNSGTCKALAHRLASSLMGRGFTEQKLAALDTV
VGNLPTDQPVIIVTTSYDGRPTDDAEEFVRWLESKRPVLQGVSYAVFGCGHHDWAKTFYR
IPILIDDLMHKAGATRLTALGTANAAVSDLFSDLELWEETNLLPALREAFPPSNSSDVES
SEPHQLQICVSKPRRVDMHRGLVEAKVTAVRTLTSPDSPEKRHVEFHVQGDTTWRPGDHV
NILPVNPLSTVSRVLAYFQLAPDHSITVNSFNTQGLPSATPVSATELFSSFVELSQPATR
KNLKALAMAAESKTDEQELIRLHDSYDALVRDKRVSVLDILERFPSISLPIGIFISMLPP
LRLRTYSLSMAPSFKPSHGSLTFSVINEPAWSGNGQYLGVGSNYLASLTPGSLLYLSPRP
AKDAFHLPADQFNTPIIMICAGSGLAPFMGFIQERMTWLKQGRPLAKGLLFFGCRGPHLD
DLYYEELSEFEDAGVVEVHRAYSRAPDDVRAKGCRHVQHRLVTEAEAVRDHWGRNAIVYV
CGSSNMARGVQTVLEEILGTLPPERYVAEIF
Conjugated Linoleic Acid (CLA) Isomerase
SEQ ID NO: 19
Cutibacterium (Propionibacterium) acnes CLA isomerase
MSSKDSRAGAGAGAAGMYAGHDYTRTDHVGGKCHSNYHGRRYMGAMGVSYDTMDRTGDKV
DGKRRHDGYVKDVRGVMAAVKGATKYGYDANGHYNKVHDMDANGCAARDWNTAGYGHDNV
AAYVKYDVTMMSAKGDWTWADGTAMHNATHARNVDTRTRDGKVHHTTDWDRSDVVTVKDY
SDADDDRYSKHYMVDACVKYTSGYVDNMRRGHVMVYYHRWADDHTTYRNHDYADKTCRMV
DDMTGHVKTWYYHVSSDYKAGWYKVGMGRRNTYAGMSGNDVCHYSKDVTRV
Fatty Acid Synthase Enzymes
SEQ ID NO: 20
Schizosaccharomyces japonicum FAS1
MVETGYTSPHLNQSVRSFTLSYGKLSPSISVPASLYSVATQLRDEFLMKYPAPSAEAIEN
DTDEVEFKQDIHLVARFIGLIAAKEEESHGHFTQLLNVFTLEFERTFLAGNEVHAVAARS
GLEIPAQKDVIKCYYRSRFLSGRTIKNHGSALLNDPKVSLGAIFGGQGYEDYFEELVELY
DVYAPFAADLVHRLAKHLLDLSQNEQAIKVFSKGLDVLAWLAGERPDRDYLVCAPVSLPL
IGLTQLVQFYVTAKILGLDVGEFAAKFSASTGHSQGIVVAAVISASTDERSFFENAKSAL
TCLFWIGVRSQLAFPTTTLPPSVLSDSLECNEGAPSPMLTVRDLPMEVLNKHIKITNTHL
PEDRKVCLSLINGPRSFVVSGPARSLYGLNLSLRKSKAGGENQARIPHSKRKLRFINRFL
SISVPFHSSYLEGARADLENDLKDISFGKLQIPVYATDDARDLRAVAPEKLPMMLAVMIT
VNVVHWEEACNFPGVTHIIDFGPGGLSGVGSMTRMNKDGRGVRIIVSDSFESIDVGSKTE
IFDRDVNSVQFAPNWLESFSPKVVKTTAGRCYVDTRMSRMLGVPPLWCAGMTPTTVSWEF
CSAIAQAGYTFELAGGGYFDPNMMREAIHKLSENIPAGSGICVNVIYINPRTYAWQIPLI
KAMVAEGYPIRGVTIAAGIPSLEVANELISDLGVQYICLKPGSTEAVNSVISIAKANPHL
PIVLQWTGGRAGGHHSFEDFHAPILSTYSSVRRCPNIVLLAGSGFGGSDDTLPYLTGEWS
DKFKLPPMPFDGVLFGSRLMVAKEARTSLGVKQAIVDAPGVEDKDWEKTYDGPAGGIVTV
LSELGEPIHKLATRGVMFWKEMDDTIFSLPRPKRLPALLAKKSKIIKKLNEDFQKVYFPA
HIVERVSAEKFKFEAVNDLAEMTYAEVLYRLIDLMYVTKDKRWIDITLRNMTGNYMRRVE
ERFTNVTGKTTIVEDFDVLSDPYTFADKFLTVYPEAKDQLINSQDAQHFFTMCANPLQKP
VPFVPAIDETFEFFFKKDSLWQSEDLDAVVGEDVGRVAILQGPMAARHSTKVNEPVKEML
DNIHNAHIAALTEKLYGSEDKVPTVEYFGGEKPLTLKKSDLEGMTFEETAGEQAVYTAPL
GSTSALPNLKSWFEILAGPEFSWRRAIFTTARIAKNWKLESNPVRRIFAPRYGQKLILKG
SGDAAVVELYETVNGIYSLSAILKYNGENIICTLTDKRNALKEHVGLDFIFNYNRAAGYC
PISEVLEGRNDRIKRFYWKVWFGNEPYPEEASITDTFTGPEVEITGSMIDDFCRVVGNQN
EVYTKRGNAKRTAPMDFAIVVGWQAITKAIFPKTIDGDLLRLVHLSNAFRMLGETPLREG
DKVKTSAVITAVINRENGKMVEVKGTLSANGTDVLEVVTRFLYRGNFTDFENTFEHTQET
PMELTLSSPKDVAVLQSKAWFQVLDPERDLSGASLTFRLNSYSRFKNKTIRASVQTKGVV
LMELPTKEVIQVASVDFQSVDCHGNPVMEFLKRQGKPIEQPVEFENGGYSLIRIMDDAYS
STFVTPPTNTPYAEVSGDYNPIHVSSVFAAFVQLPGVHGITHGMFNSAATRRFVETFAAQ
NVPDRVKQYEVKFVNMALPNSELVTKLSHVGMINGRKIIKIQVVNKETEEVVIVGSAEVE
QPVTAYVFTGQGSQEQNMGMDLYSTSPVARAIWDSADNHFLSNFGFSILDIVRNNPKNLT
IHFGAAKGQFIRKNYMDMIYEKANEDGTTSIVPVFPTITEESTSYTFTHPQGLLSATQFT
QPALTLMEKAAFEDMRSRGLVQQNSVFAGHSLGEYSALSSMGDVMTIEDLVDLVFLRGLT
MQNAVHRDAEGRSEYGMVAASPLRVSPTFNEAGLRFIVDHIAQQLKLLLEIVNENVENQQ
YVVSGHLHSLSTMGNVLNFLKVQKIDFQKLVETLSMEQLKAELSEIVSKCHEKTLEELKK
NNGIIELQRGYATIPLKIDVPFHSTFLRGGVRMFREYLMKRITPHQVNISKLRHKFIPNL
TARPFDVTKEYFQEVYELTGSQRIKKIIQNWDTYASS
SEQ ID NO: 21
Schizosaccharomyces japonicum FAS2
MRPEVEQELAHTLLLELLAYQFASPVRWIETQDVLLSPPYSSERLVEIGPSPTLAGMAQR
TLKMKYENIDAALSMNREVLCYSRDKKNIYYNIENEEPQEVAPEPVAAAAPTPAAAAPAA
PAAAPAPAASEPVAAAAELPDELPKAVDVLHILVAQKLKKTVDEVSPQKAIKDLVGGKST
LQNEILGDLQKEFGSTPEKPEETPLDELGATMQTSFSGQLGKQSSSLISRMISSKMPGGF
SNSAVRAYLKNRFGLATGRTNSVLLLGLTMEPAARIGSEAEAKAWLDTVAQKYASRNGIT
LSAPAAASSSGGGQAAVIDEATFKKLTQSNTKLVTQQLELFANYLNKDLRAGDKAAVAER
AVSDALRAQLDLWNNEHGEFYASGISPIFSPLKARVYDSEWNWARQDALKMFYDIIFGRL
KLVDREIVSRCIAVMNRANPVLLEFMQYHIDHCPSDKGATYQLAKELGQQLIDNCKEAIE
KPPVFKDVNYPTAPSTEIDERGNLNYEEVPRSGVRKLEQYVAEMARGGKVPPASKSKAKV
QDDLARIYRIITSQNKMTRSSKLQVKQLYAQVLRNLNIRLPSGNDKTPAKETIPFLHLCR
KSGEAWEFNKTLTAAYLDVLENGAKNGITFHGKCALVTGAGAGSIGVQIVKGLLAGGARV
IVTTSRYSRKVTEFYQSLFTRYGSRGSSLIVVPFNQASKQDVEALVKYIFDEKDGLGWNL
DYIVPFAAIPENGREIDNIDSRSELAHRIMLTNVMRLLGAVKVEKAARGFDTRPAQVILP
LSPNHGTFGNDGMYSESKLALEALFNRWSSESWSNYLLICGAVIGWTRGTGLMAPNNIVA
QGIEKYGVRTFSQAEMAFNILGLMSQKIVNICQTEPVYANLNGGLELLPSIKDLSTRLRT
ELIQTAEIRKAVAAEAAFDHNVLNGPGSDAIYHKTAIQPRANLKFEFPKAKPYEAYAHLS
HLRGMVDLERVAVVTGFSEVGPWGNSRTRWDMECFGEFSLEGCVELAWIMGLIKNFNGTL
KNGKPYSGWVDAKTGEPVDDKDVKSKYESYMIEHCGIRIVESDMFNDYHPEKKELLQEVV
IDHDLEPFEASKDAAHEFKLKHGDKVEIFAIPDSTEWSVRFKRGTSLLVPKALRFDRFVA
GQIPRGWDPKRYGIPEDIVSQVDPTTLYVLVSTVEALVSSGITDPYECYKYIHVSELGNA
VGSGVGGMSALRGMYKDRWCDKPVQKDILQESFINSASAWINMLLLSSSGPIKTPVGACA
TAVESVDAAVEMISSGKAKICIAGGFDDFSEEGSYEFANMGATSNSIKETERGRTPAEMS
RPATTTRDGFMEAQGAGVEIVMQAKLAIEMGVPIYGIIGYANTAMDKQGRSVPAPGKGVL
TGARELQSKFPSPLLDIKYRARQLKLRRQQISDWAEQEYLYLDEELSAIKSQDPSVNIDD
YRLERVAHIKKEIARQDKEALATFGNDFWRKDPSIAPIRGALAVWGLGVDDIGVASFHGT
STVANEKNECDVLDSQLKHLNRSKGNPILGVFQKYLTGHSKGGAGAWMLNGVLQILQTGY
VPGNRNADNIDAYMERFDHVMFPSQGIQTDGVRAGSVTAFGFGQVGGQIIAIHPDYVLAT
LDKDTYEAYIAKTKIRYQASYRYTHDALVNNNLFRAKDHPPYTAEQEKIVYLNPEARAYL
DEKTNTYKYASGKPKPKKQPASVSKTASMLDSMTKALAGSSQTVGVDVELIAAINVSNDT
FLERNFTEAERSYCSSTPNPQASYAGRWSAKEAVFKSLGVKGQGAGASLKDIEIVASESG
APTVQLHGAAKEAADNAGVKNVSVSISHDDIQSVSVAIAEH
OLE1 Homologs
SEQ ID NO: 22
Rhodotorula toruloides
MTASSALETSLPHSVGPEAATTTAKPPRAPLRMRHPDYTQTDVLDSSDSDAASDSEGETT
AVDDGTYEDDNYVRKVLSKEKPLPPITWKNIHRNIQWISTLALTIVPLLAIYGAFTTPLK
WQTAVWSVVYYYYTGLGITAGYHRLWAHRSYTASLPLQYFLALGGSGAVEGSVKWWSRGH
RAHHRYTDTDLDPYSAQKGFWWAHLGWMIVKPRRRPGVADVSDLNNNPVVKWQHRYYLPL
ILGMGFVFPTIVAGLGWGDFRGGFFFAGAARLLFVHHSTFCVNSLAHWLGETPFDDKHTP
KDHWLTALATVGEGYHNFHHEFPSDYRNALRWWQYDPTKLFIWTMSKLGLASQLKTFPDN
EIKKGQYAMTLKAVAREAENIEWPKSSNHLPVLTWDEFQDACKTRQLLVVAGFIHDVSTF
IDQHPGGAGLIKTRLGRDATTAFYGGYYDHSNGAANLLAQYRVGVIEGGYEVEHMKKYSE
VVENLKKHGADGVAGKSADLVKGPKQTSVIKGDPQLKSAPLETLAKPPTFSETNLLGGLS
LKVKA
SEQ ID NO: 23
Rhodotorula graminis
MRHPDYSQGDVVESSDSDREASDSEGETTAVDDGTYQDDNFVRKVLAKERPLPPITLKTL
PQNINVISTLALTVVPALAIYGAFTTQIKWQTALWSVIYYFYTGLGITAGYHRLWAHRSY
TASLPLQYFLALGGSGAVEGSIKWWSRGHRAHHRYTDTDLDPYSAQKGFWWSHIGWMVVK
PRRTPGVADVSDLSVNEVVKWQHRWYVYLIVGMGFVFPTLVAGLGWGDYRGGFFFAGAAR
LLFVHHSTFCVNSLAHWLGETPFDDKHSPRDHWITALVTVGEGYHNFHHEFPQDFRNAIQ
TFQYDPTKWFIIVMHWLGLASQLKTFPDNEIRRGQYAMKLKAVAREADEIRWPKSSNHLP
VLTWDEFQEACKTRQLMVISGYIHDVSTFIDEHPGGRALIKTRLGRDATNAFYGGYYDHS
NGANNVLAQYRVGVIEGGYEVEHLKRFSKLIEDLKESGADGVAGKSADLHGGAKSSTRTT
VIKGDPQLKSAPLADVAQPPTFKNVRLTGGLGHAIYA
SEQ ID NO: 24
Rhodotorula diobovata
MAAAQPTTAVNAALATALPHAVDSATTTAKPPRAPLRMRHPNYAEGDVVESSDSDREATD
SEGETTAVDDGTYQDDNYVRKVLAKERPLPPITLKTLPQNINVISTLALTVVPALAIYGA
FTTQIRWQTALWSVIYYFYTGLGITAGYHRLWAHRSYTASLPLQYFLALGGSGAVEGSIK
WWSRGHRAHHRYTDTDLDPYSAQKGFWWSHIGWMIVKPRRTPGVADVSDLSANEVVKWQH
RWYVYLIVGMGFVFPTLVAGLGWGDYRGGFFFAGAARLLFVHHSTFCVNSLAHWLGETPF
DDKHSPRDHWITALVTVGEGYHNFHHEFPQDFRNAIQTFQYDPTKWFIIVMHWLGLASQL
KTFPDNEIRRGQYAMKLKAVAKEADEIRWPKDSNHLPVLTWDEFQEACKTRQLMVISGYI
HDVSTFIDEHPGGRALIKTRLGRDATNAFYGGYYDHSNGANNVLAQYRVGVIEGGYEVEH
LKRFSKLIEDLKESGADGVAGKNADLQSGPARVTVIKGDPQLKSAPLADVAKPPTFKNVR
LTGGLGHPIEA
SEQ ID NO: 25
Rhodotorula mucilaginosa
MAMAAEHAVTSALATSLPHSVGPEAAAKPPRAPLRMRHPDMTNMPDSSDSDHDASDSEGE
TTAVDDGTYNDDNYVRKVLAKEKPLPPITLGNLYKNINVISTLALTVVPALAIYGAFTTP
VMWQTALWAVAYYFYTGLGITAGYHRLWAHRAYTASLPLQYFLALGGSGAVEGSIKWWSR
GHRAHHRYTDTDLDPYSASKGFWWAHVGWMIVKPRRRPGVADVSDLSVNPVVQWQHRWYL
PLIVGMGFVFPTVVAGLGWGDWRGGFFFAGAARLLFVHHSTFCVNSLAHWLGETPFDDKH
SPKDHWVTALVTIGEGYHNFHHEFPQDYRNATKFYQWDPTKLFIRTASLLGLASQLKTFP
DNEIRRGQFAMKLKAISRQAEEIQWPKDSNHLPVLTWDDFEAECKTRQLMVIGGFIHDVS
TFIDEHPGGRALIKTRLGRDATTAFYGGVYDHSNGASNVLSRYRVGVISGGYEVEHMK
SEQ ID NO: 26
Cryptococcus gattii
MSAAVLPMTPPLEKEESELVHRRPSPADELTPPHTPEQNPCKSTNGKGKDPIDPSLLPSD
RHIPDNYVSYTIANQKYLPPITWKNLIHNIQWISFLALTVTPSLAIYGIFTTTWNTKTAI
WSVIYYFITGLGITAGYHRLWAHRAYNASIPLQYVLATAGSGAVEGSIKWWCRGHRAHHR
YTDTDLDPYSAEKGFFWSHVGWMLVKPRGKIGVADVSDLSKNRVVKWQHRNYIPLILGMG
FVFPTVVAGLGWGDWRGGFFFAGAARLCFVHHSTFCVNSLAHWLGEQPFDNKHSPRDHII
TALCTIGEGYHNFHHQFPQDFRNAIKWFQYDPTKWFIWTMSTLGLASHLKRFPDNEVKKG
QYTMKLQLLKEQADELQWPKSSNDLPVISWDDFKAEAKERSLVAIHGFIHDCSSFVEDHP
GGAHLIKRAIGTDATTAFFGGVYDHSNAAHNLLAMMRVGILDGGMEVEHLKRRPAESAAS
SIINSPVSSASASSVDIQSLADDDFRLDQTQLNSQGPKPKAPFGQPQAQVADRWTLSVPP
SEKLRIVQTVPEIRPGLLTHRSVGKLDKVTKADVGGEANEFSGNEPVAAA
SEQ ID NO: 27
Cryptococcus neoformans
MSAAVPPMTPPFEKEASELVHRRPSPVDQLDPAFTPPHTPEQNPCKSTKGKGIDPIDPTL
LASDRHIPDNYVSYTIANQKYLPPITWKNLIYNIQWISFLVLTVTPSLAIYGVFTTAWNT
KTAIWSVIYYFITGLGITAGYHRLWAHRAYNAGIPLQFAFAIAGSGAVEGSIKWWCRGHR
AHHRYTDTELDPYSAEKGFFWSHVGWMLVKPRGKIGVADVSDLSRNRVVKWQHRNYIPLI
LGMGFVVPTVVAGLGWGDWRGGFFFAGAARLCFVHHSTFCVNSLAHWLGEQPFDNKHSPR
DHIITALCTIGEGYHNFHHQFPQDFRNAIKWFQYDPTKWFIWTMSQLGLASHLKRFPDNE
VKKGQYTMKLQLLKEQADQLEWPKSSNDLPVISWDDFKAEAKERSLVAIHGFIHDCSSFV
EDHPGGAHLIKRAIGTDATTAFFGGVYDHSNAAHNLLAMMRVGILDGGMEVEHLKRRPAE
SAASSVTNSPVSSASASSVDIQSLADDDFRLDQTQLNSQGPKPKAPFGQPQAQVADRWTL
SVPPSEKLRIVQTVPEIRPGLLTHRQAGKLDKVTKADVGGEVIEFIGEKPVAAA
SEQ ID NO: 28
Vanrija humicola
MSAAAVSQPPPKVVNELDPEADDFQTPDNYVTKTVQNQKRLPPITWRNLLSNIQWISTLA
LTVPPALAIYGLMNYPLQWKTLVWSIVYYFITGLGITAGYHRLWAHRSYNASKPLQVALA
LAGAGSVQGSIKWWSRGHRAHHRYTDTKLDPYSAHEGFWWAHMGWMLVKPRGKIGVADIS
DLNKSKVVRWQHNNYVLIMVFMGLIFPTLVAGLGWGDWAGGYFFAGAARLVFVHHSTFCV
NSLAHWLGETPFDNKHTPKDHFITALVTVGEGYHNFHHQFPMDFRNAIKWYQYDPTKWFI
WTMSKLGLASHLKKFPDNEIKKGQYTMKLQVLQEQSKDIKWPVHSNDLPVISWEDFKAEA
KERSLVAIHGFIHDVSSFIEDHPGGAHLIKKAIGTDATTAFFGGVYDHSNAAHNLLAMMR
VGILDGGMEVEHLKLQGLRRNLSSASLESGVSSASSVSVQSILSEVENIENHKPEYQPVA
MVSNPYTFSIPPSEKLRIIQSTPEVRPTVLSRLNSQANSAAPSRPISPPLSP
SEQ ID NO: 29
Cutaneotrichosporon curvatum
MSASTATAPPATAPAVANPTPAAASAAAAAPAATKDKAETIDPESEHFVVSQNYVTRTVE
NMTMLPPVTWSNLLQNIQWISFTALTVPPAMATYGLCTLELQRKTVIWAIVYYFMTGLGI
TAGYHRLWAHRAYNASAPLQYFLALCGAGSVQGSIKWWSRGHRAHHRYTDTKLDPYSAHE
GFWWAHVGWMLVKPRGKIGVADISDLSRNPVVKWQHNNYVMLMVLMGLVFPTLVAGLGWG
DWKGGLLFAGAARLVFVHHSTFCVNSLAHWLGETPFDNKHTPKDHFITALVTVGEGYHNF
HHQFPMDFRNAIKWYQYDPTKWFIWTMSNVGLASHLKKFPDNEIKKGQYTMKLQMLQEQS
GSIQWPKHSNDLPVISWEDFQAEAKERSLVAIHGFIHDCSSFLEDHPGGIHLIKKAIGTD
ATTAFFGGVYDHSNAAHNLLAMMRVGILDGGMEVESLKLENLQRSMSVSSMESDAASSAS
SVSVSSISPRWPH
SEQ ID NO: 30
Filobasidium floriforme
MADTAFAAMHRKAPTNPVPIPQEEKSETKLGVKPLEPTDIPSDLDVPDNYVTRTIETQKR
LPPVTMANWHKNIQWVSFLALTVTPALTIYGLFNVKWNTYTAIWSVVYYFVTGLGITAGY
HRLWAHRSYTASRPLEYILACAGAGAVQGSIKWWSRGHRAHHRYTDTSLDPYSAHEGFWW
AHVGWMIFKPRTKIGVADISDLTRSKVVRWQHNNYLSLLLVMGLFFPMGVAGLGWGDWKG
GFFFAGAARLLFVHHSTFCVNSLAHWLGEHTFDDKHTPRDHFITALVTIGEGYHNFHHQF
PMDYRNAIKWNQYDPTKWFIATAQLLGLASHLKKFPDNEIKKGIYTMQLQKLAQAGEVIE
WPTDSNHLPVVSWDDFVDESKNRALIAVHGFIHDVSSFMDDHPGGQHALKKFIGKDATTA
FYGGVYDHSHAAQNLLAMMRVGCLEGGMEVEHLKQKVRERSLSGSSANSAFSSNEDVASL
ASYSSGRLSDGELSDVDFLQDLPKKEKPKHQPGCYVPDKFTLAIPPSEEFKIIRSAPRLR
KTGGGHGSMASMTSLEDVSRADKIGDVAPLVAAAA

1. A microbial cell for producing milk fat triglycerides, wherein the cell expresses a biosynthetic pathway comprising at least one heterologous enzyme, the biosynthetic pathway producing triglycerides having short chain fatty acids esterified at sn-3, and wherein the cell produces triglycerides having at least about 5% C4 and C6 fatty acids esterified at sn-3 on a molar basis.

2. The microbial cell of claim 1, wherein the cell produces triglycerides having at least about 10% C4 and C6 fatty acids esterified at sn-3 on a molar basis.

3. (canceled)

4. The microbial cell of claim 1, wherein;
the triglycerides further comprise C12 to C18 fatty acids esterified at sn-3;
the triglycerides comprise C4:0, C6:0, C8:0, and C10:0 fatty acids esterified at sn-3; and/or
the triglycerides have predominately C12 to C18 fatty acids esterified at sn-1 and sn-2 on a molar basis.

5.-9. (canceled)

10. The microbial cell of claim 1, wherein the biosynthetic pathway comprises one or more heterologous fatty acyl-CoA synthetase (ACS) having specificity for short chain fatty acid substrates.

11. The microbial cell of claim 10, wherein at least one heterologous ACS comprises an amino acid sequence that is at least 70% identical to an amino acid sequence selected from SEQ ID NOs: 1-7.

12. (canceled)

13. The microbial cell of claim 11, wherein the microbial cell expresses a heterologous ACS comprising an amino acid sequence that is at least about 80% identical to the amino acid sequence of SEQ ID NO: 2 or SEQ ID NO: 3.

14. (canceled)

15. The microbial cell of claim 1, wherein the biosynthetic pathway comprises a heterologous diacylglycerol O-acyltransferase (DGA) having specificity for short chain fatty acid CoA substrate.

16. The microbial cell of claim 15, wherein the DGA comprises an amino acid sequence that is at least 70% identical to an amino acid sequence selected from SEQ ID NOs: 8-17.

17.-18. (canceled)

19. The microbial cell of claim 1, wherein the microbial cell expresses a conjugated linoleic acid (CLA) isomerase.

20. The microbial cell of claim 19, wherein the CLA isomerase comprises an amino acid sequence that is at least about 75% identical to an amino acid sequence selected from SEQ ID NO: 19.

21. The microbial cell of claim 1, wherein the microbial cell comprises a modification that results in decreased amount or activity of one or more endogenous diglyceride acyltransferases.

22. (canceled)

23. The microbial cell of claim 1, wherein the cell has one or more genetic modifications that reduce C16:1 fatty acid production and/or increase C18:1 fatty acid production.

24.-25. (canceled)

26. The microbial cell of claim 23, wherein the microbial cell expresses at least one heterologous OLE1 enzyme converting C18:0 to C18:1, and which is optionally selected from an enzyme that comprises an amino acid sequence having at least 80% sequence identity to one of SEQ ID NOS: 22 to 30.

27. The microbial cell of claim 23, wherein the microbial cell has a deletion, inactivation, or reduced expression or activity of one or more fatty acid elongase enzymes.

28. The microbial cell of claim 1, wherein the microbial cell expresses one or more heterologous fatty acid synthases (FAS), optionally where the heterologous FAS enzymes produce one or more of C10:0, C12:0, and C14:0 fatty acids.

29. The microbial cell of claim 28, at least one heterologous fatty acid synthase comprises a subunit comprising an amino acid sequence that is at least 70% identical to the amino acid sequence of SEQ ID NO: 20 (FAS1), and a subunit comprising an amino acid sequence that is at least 70% identical to the amino acid sequence of SEQ ID NO: 21 (FAS2).

30. The microbial cell of claim 1, further comprising a biosynthetic pathway producing one or more lactones.

31.-38. (canceled)

39. The microbial cell of claim 1, wherein the cell produces β-carotene through a heterologous biosynthetic pathway.

40.-43. (canceled)

44. The microbial cell of any one of claims 1 to 43, wherein the microbial cell is a yeast or fungal cell or a bacterial cell.

45.-46. (canceled)

47. The microbial cell of claim 44, wherein the yeast or fungal cell is Yarrowia lipolytica or Yarrowia phangngensis.

48.-52. (canceled)

53. A method for making a composition comprising milk fat triglycerides, comprising: culturing a microbial cell of claim 1 in the presence of C4:0, C6:0, C8:0, C10:0, C12:0, and C14:0 fatty acid substrates, and recovering the composition from the culture.

54.-60. (canceled)

61. A composition comprising milk fat triglycerides produced according to the method of claim 1.

62.-63. (canceled)


SEQUENCE LISTING (30 sequences)

SEQ ID NO: 1 - protein, 566 aa, Escherichia coli
MHPTGPHLGPDVLFRESNMKVTLTFNEQRRAAYRQQGLWGDASLADYWQQTARAMPDKIAVVDNHGASYTYSALDHAASCLANWMLAKGIESGDRIAFQLPGWCEFTVIYLACLKIGAVSVPLLPSWREAELVWVLNKCQAKMFFAPTLFKQTRPVDLILPLQNQLPQLQQIVGVDKLAPATSSLSLSQIIADNTSLTTAITTHGDELAAVLFTSGTEGLPKGVMLTHNNILASERAYCARLNLTWQDVFMMPAPLGHATGFLHGVTAPFLIGARSVLLDIFTPDACLALLEQQRCTCMLGATPFVYDLLNVLEKQPADLSALRFFLCGGTTIPKKVARECQQRGIKLLSVYGSTESSPHAVVNLDDPLSRFMHTDGYAAAGVEIKVVDDARKTLPPGCEGEEASRGPNVFMGYFDEPELTARALDEEGWYYSGDLCRMDEAGYIKITGRKKDIIVRGGENISSREVEDILLQHPKIHDACVVAMSDERLGERSCAYVVLKAPHHSLSLEEVVAFFSRKRVAKYKYPEHIVVIEKLPRTTSGKIQKFLLRKDIMRRLTQDVCEEIE

SEQ ID NO: 2 - protein, 548 aa, Pseudomonas putida
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 3 - protein, 508 aa, Rhodopseudomonas palustris
MSFAYFDWIAHHAEVRPERIAVVDLASSRKISYRAMDERVDRLAAHLAALGVGRGDRVAVLALNAVETLEVQFACFRLGAIFVPLNVRLTVHELSYIVGDAAPRVLAHDDELAPMAKELKAACSVPHLLAFGAAYEAALAASPRLGASEPVTLDDVSTIMYTSGTTGKPKGAMITHGMTFINAVNLGIPAFISQRTVFLCVLPLFHTGGLNCYTNPVLHAGGTTLLMRAFDPGAALSIIGDPSVGLTHFFGVPSIYQFMCQHPAFAATDLSRLQIAGVGGAPMPVPLLKIWQERGCALVQGYGMTETSPAVLMLDADDAARKAGSAGKPVLHADLKIVGPDGDPVKPGEMGELWVKGPNITPGYWNRPDANRTSFTDGWLHTGDAARVDDEGFYYIVDRTKDMYISGGENVYPAEVEDVLYQLPEIAEAAVIGAPDPQWGETGVAVVALKPGQELSEAKLLAHCRERLARFKCPQRVSFVEALPRNATGKVHKPTLRERILVRETADA

SEQ ID NO: 4 - protein, 534 aa, Rhodopseudomonas palustris
MTSLEATGGVPGPGRIGRVAIGDILRKSARRFPDRVALTDGGRSVTYTELERDANRFANALVARGLKPGAKISTVCNNSIEFVKALFGIHRAGLVWVPINTMLGPDDMGYILDHAGVKVAVIDDNLHGQPERRAALEARGIDLIAINLAGKAADTGLPVFDQLIEGLSEIEPDVAFDDRDLAMIIYTSGTTSRPKGAMHCHLAVTMAVMSNAIEMQLSRKDGITGQFPLFHCAAHVLLLSYLIVGGQMAIMRGFDPVACMEAIQRNKLTVFIGLPLMYQVILDHPRRKEFDLSSLRCCIYTMAPMPRPLLERAIAELCPTFVQPSGQTEMYPATTMSQPDRQLARFGNYWGESTLVNETAIMDDAGNLLPPGEVGEIVHRGPNVMLGYYKDPEATEAARKFGWHHTGDLALIDEHGEVLFLDRKKDMIKSGGENVASIKIEETLLAHPSVMNAAVVGLPHPQWGEAVSGFVKLKPGASATEAEIVEHCKKHLGGFQVPKLLRIVDEMPMTATGKLRKVELRNQFTDHFMLGQTG

SEQ ID NO: 5 - protein, 721 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNMGALLEKRGKEFLGVKYKDPISSFSHFQEFSVRNPEVYWRTVLMDEMKISFSKDPECILRRDDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 6 - protein, 660 aa, Arabidopsis thaliana
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 7 - protein, 744 aa, Saccharomyces cerevisiae
MAAPDYALTDLIESDPRFESLKTRLAGYTKGSDEYIEELYSQLPLTSYPRYKTFLKKQAVAISNPDNEAGFSSIYRSSLSSENLVSCVDKNLRTAYDHFMFSARRWPQRDCLGSRPIDKATGTWEETFRFESYSTVSKRCHNIGSGILSLVNTKRKRPLEANDFVVAILSHNNPEWILTDLACQAYSLTNTALYETLGPNTSEYILNLTEAPILIFAKSNMYHVLKMVPDMKFVNTLVCMDELTHDELRMLNESLLPVKCNSLNEKITFFSLEQVEQVGCFNKIPAIPPTPDSLYTISFTSGTTGLPKGVEMSHRNIASGIAFAFSTFRIPPDKRNQQLYDMCFLPLAHIFERMVIAYDLAIGFGIGFLHKPDPTVLVEDLKILKPYAVALVPRILTRFEAGIKNALDKSTVQRNVANTILDSKSARFTARGGPDKSIMNFLVYHRVLIDKIRDSLGLSNNSFIITGSAPISKDTLLFLRSALDIGIRQGYGLTETFAGVCLSEPFEKDVGSCGAIGISAECRLKSVPEMGYHADKDLKGELQIRGPQVFERYFKNPNETSKAVDQDGWFSTGDVAFIDGKGRISVIDRVKNFFKLAHGEYIAPEKIENIYLSSCPYITQIFVFGDPLKTFLVGIVGVDVDAAQPILAAKHPEVKTWTKEVLVENLNRNKKLRKEFLNKINKCTDGLQGFEKLHNIKVGLEPLTLEDDVVTPTFKIKRAKASKFFKDTLDQLYAEGSLVKTEKL

SEQ ID NO: 8 - protein, 489 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKAANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEYLVSIPLRMFRLWAFTGMMAQIPLAWIVGRFFRGNYGNAAVWLSLIIGQPVAVLMYVHDYYVLNREAPAAGT

SEQ ID NO: 9 - protein, 489 aa, Capra hircus
MGDRGGAGGSRRRRTGSRPSIQGGSRPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKKANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCLRHFYKPMLRRGSSKWAARTGVFLASAFFHEYLVSIPLRMFRLWAFTGMMAQIPLAWIVGRFFRGNYGNAAVWLSLIIGQPVAVLMYVHDYYVLNREAPTAGT

SEQ ID NO: 10 - protein, 489 aa, Ovis aries; identical to SEQ ID NO: 9

SEQ ID NO: 11 - protein, 489 aa, Bubalus bubalis
MGDRGGAGGSRRRRTGSRPSIQGGSGPAAAEEEVRDVGAGGDAPVRDTDKDGDVDVGSGHWDLRCHRLQDSLFSSDSGFSSYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFPKLFSYRDVNLWCRERRAGAKAKAALAGKKANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCPNAVAELMQFGDREFYRDWWNSESITYFWLNWNIPVHKWCIRHFYKPMLRRGSSKWAARTAVFLASAFFHGYLVSIPLRMFRLWAFTGMMAQIPLAWIVGRFFRGNYGNAAVWLSLIIGQPVAVLMYVHDYYVLNREAPAAGT

SEQ ID NO: 12 - protein, 361 aa, Bos taurus
MKTLIAAYSGVLRGTGSSILSALQDLFSVTWLNRSKVEKQLQVISVLQWVLSFLVLGVACSVILMYTFCTDCWLIAVLYFTWLVFDWNTPKKGGRRSQWVRNWAVWRYFRDYFPIQLVKTHNLLTSRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITIPRLERPTQQDIDLYHAMYVQALVKLFDQHKTKFGLPETEVLEVN

SEQ ID NO: 13 - protein, 361 aa, Ovis aries
MKTLIAAYSGVLRGTGSSILSALQDLFSVTWLNRSKVEKQLQVISVLQWVLSFLVLGVACSAILMYAFCTDCWLIAVLYFTWLVFDWNTPKKGGRRSQWVRNWAVWRYFRDYFPIQLVKTHNLLTSRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLRNRKGFVKLALRHGADLVPIYSFGENEVYKQVIFEEGSWGRWVQKKFQKYIGFAPCIFHGRGLFSSDTWGLVPYSKPITTVVGEPITIPKLEHPTQQDIDLYHAMYMEALVKLFDQHKTKFGLPETEVLEVN

SEQ ID NO: 14 - protein, 339 aa, Bos taurus
MAMAFLSQLNLQEILQTLSVLQWMPVYVFLGAIPIIVIPYFLVFTKFWMVSVLALAWLAYDWNTHSQGGRRSAWVRNWTIWKYFQNYFPIKLVKTHDLSPRHNYIIASHPHGVLPYGTFINFATETTGFARIFPGITPYVATLEGIFWIPIVREYVMSMGVCPVSELALKYLLTQKGSGNAVVIMVGGGAEALLCHPGATTVLLKQRKGFVKVALETGAYLVPSYSFGQNEVHNQETFPEGTWKRFFQKALQDTLKKLLRLSVCTFHGRGLTRGSWGFLPFNHPITTVVGEPLPIPRIKKPNEETVDKYHALYINALQKLFDEHKVQYGLSETQELTII

SEQ ID NO: 15 - protein, 339 aa, Capra hircus
MAMAFLSQLNLQEILQTLSVLQWVPVYIFLGAIPIILIPYFLVFTKFWMVSVLALAWLAYDWNTHSQGGRRSAWVRNWTIWKYFRNYFPVMLVKTHDLSPRQNYIIASHPHGILPYGIFINFATETTGFARIFPAITPYIATLEGIFWIPIVREYVMSMGVCPVSELALKYLLTQKGSGNAVVIVVGGAAEALLCHPGASTVFLKQRKGFVKVALKTGAYLVPSYSFGQNEVHNQETFPEGTWKRFFQKTLQNTFKKILGLNFCTFHGRGLIRGSWGFLPFNHPITTVVGEPLPIPRIKKPNKETVDKYHALYINALRKLFDEHKVQYGLSETQELTIV

SEQ ID NO: 16 - protein, 424 aa, synthetic construct
MAAHRTSSPVHLKLAESPLSSRNIFKQNHEGLFNLCMVTLVAVIIRLFLENLLKYGWLMKRDFWLSTFTAWPLFICSLGLPIFPLAAFVVEKLAQKNLLPEPIVLCSHVIITSASVLYPALVILRFDCALMSGIGLMLYSCALWLKLVSYAHTSYDMRCEAKSRLEGKSSADSKNGELPYRVNIKDLAYFMVAPTLCYQLSYPRTQFIRKFWVARQVLKLILVNVVMGFIIEQYMIPVMHNSKPPRRGYWLHFIERNLKLAVPSIGLWFCIFYSIFHLWLNIVAELLRFGDREFYKDWWNAKNMEEYWKMWNIPVHRWMVRHLYGPCMKRKLPRWVAISISFLLSAVLHEICVSVPCHVFQLWAFNGMMLQIPLVLSSKPLQKRFPSSKAGNVFFWFLFCIYGQPNCVLMYYHALMERRGLRID

SEQ ID NO: 17 - protein, 363 aa, Euonymus alatus
MMDAHQEIKNFIKVWVQAMVCLSYAYYFSSRLPKGLLRLLSLLPVLYLLLIAPLNISSFILSSITGFFLAWLTTFKVISFAFDQGPLYPLPQNLLHFISIACLPITIKRNPSPKLKSTTNPSPISHLLKKAFMSFPSKVLFHWVIAHLYQYKKYMDPNVVLVIYCCHVYVMLDISLSLCATLAEFLCGFDVEPQFKEPYLATSLQDFWGRRWNIIVSSVLRSTVYAPTRNIASYLIGSRWAYFPAIIATFVVSGVMHDVVYYVYMMHMYPKWDMTGHFVLHGICEALEVEMKCKRSRSDKWRRHPAVDWVMVMGFVMGTSVSLLFVPLLRDNVDQIVAEEYSILFNFVREKIVMLGTRFVCGN

SEQ ID NO: 18 - protein, 1051 aa, synthetic construct
MAIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRRGQSATGLSQGSMSASGATSSVASPGPPAATGAQSNPAGGQRISFFYGSNSGTCKALAHRLASSLMGRGFTEQKLAALDTVVGNLPTDQPVIIVTTSYDGRPTDDAEEFVRWLESKRPVLQGVSYAVFGCGHHDWAKTFYRIPILIDDLMHKAGATRLTALGTANAAVSDLFSDLELWEETNLLPALREAFPPSNSSDVESSEPHQLQICVSKPRRVDMHRGLVEAKVTAVRTLTSPDSPEKRHVEFHVQGDTTWRPGDHVNILPVNPLSTVSRVLAYFQLAPDHSITVNSFNTQGLPSATPVSATELFSSFVELSQPATRKNLKALAMAAESKTDEQELIRLHDSYDALVRDKRVSVLDILERFPSISLPIGIFISMLPPLRLRTYSLSMAPSFKPSHGSLTFSVINEPAWSGNGQYLGVGSNYLASLTPGSLLYLSPRPAKDAFHLPADQFNTPIIMICAGSGLAPFMGFIQERMTWLKQGRPLAKGLLFFGCRGPHLDDLYYEELSEFEDAGVVEVHRAYSRAPDDVRAKGCRHVQHRLVTEAEAVRDHWGRNAIVYVCGSSNMARGVQTVLEEILGTLPPERYVAEIF

SEQ ID NO: 19 - protein, 291 aa, synthetic construct
MSSKDSRAGAGAGAAGMYAGHDYTRTDHVGGKCHSNYHGRRYMGAMGVSYDTMDRTGDKVDGKRRHDGYVKDVRGVMAAVKGATKYGYDANGHYNKVHDMDANGCAARDWNTAGYGHDNVAAYVKYDVTMMSAKGDWTWADGTAMHNATHARNVDTRTRDGKVHHTTDWDRSDVVTVKDYSDADDDRYSKHYMVDACVKYTSGYVDNMRRGHVMVYYHRWADDHTTYRNHDYADKTCRMVDDMTGHVKTWYYHVSSDYKAGWYKVGMGRRNTYAGMSGNDVCHYSKDVTRV

SEQ ID NO: 20 - protein, 2077 aa, synthetic construct
MVETGYTSPHLNQSVRSFTLSYGKLSPSISVPASLYSVATQLRDEFLMKYPAPSAEAIENDTDEVEFKQDIHLVARFIGLIAAKEEESHGHFTQLLNVFTLEFERTFLAGNEVHAVAARSGLEIPAQKDVIKCYYRSRFLSGRTIKNHGSALLNDPKVSLGAIFGGQGYEDYFEELVELYDVYAPFAADLVHRLAKHLLDLSQNEQAIKVFSKGLDVLAWLAGERPDRDYLVCAPVSLPLIGLTQLVQFYVTAKILGLDVGEFAAKFSASTGHSQGIVVAAVISASTDERSFFENAKSALTCLFWIGVRSQLAFPTTTLPPSVLSDSLECNEGAPSPMLTVRDLPMEVLNKHIKITNTHLPEDRKVCLSLINGPRSFVVSGPARSLYGLNLSLRKSKAGGENQARIPHSKRKLRFINRFLSISVPFHSSYLEGARADLENDLKDISFGKLQIPVYATDDARDLRAVAPEKLPMMLAVMITVNVVHWEEACNFPGVTHIIDFGPGGLSGVGSMTRMNKDGRGVRIIVSDSFESIDVGSKTEIFDRDVNSVQFAPNWLESFSPKVVKTTAGRCYVDTRMSRMLGVPPLWCAGMTPTTVSWEFCSAIAQAGYTFELAGGGYFDPNMMREAIHKLSENIPAGSGICVNVIYINPRTYAWQIPLIKAMVAEGYPIRGVTIAAGIPSLEVANELISDLGVQYICLKPGSTEAVNSVISIAKANPHLPIVLQWTGGRAGGHHSFEDFHAPILSTYSSVRRCPNIVLLAGSGFGGSDDTLPYLTGEWSDKFKLPPMPFDGVLFGSRLMVAKEARTSLGVKQAIVDAPGVEDKDWEKTYDGPAGGIVTVLSELGEPIHKLATRGVMFWKEMDDTIFSLPRPKRLPALLAKKSKIIKKLNEDFQKVYFPAHIVERVSAEKFKFEAVNDLAEMTYAEVLYRLIDLMYVTKDKRWIDITLRNMTGNYMRRVEERFTNVTGKTTIVEDFDVLSDPYTFADKFLTVYPEAKDQLINSQDAQHFFTMCANPLQKPVPFVPAIDETFEFFFKKDSLWQSEDLDAVVGEDVGRVAILQGPMAARHSTKVNEPVKEMLDNIHNAHIAALTEKLYGSEDKVPTVEYFGGEKPLTLKKSDLEGMTFEETAGEQAVYTAPLGSTSALPNLKSWFEILAGPEFSWRRAIFTTARIAKNWKLESNPVRRIFAPRYGQKLILKGSGDAAVVELYETVNGIYSLSAILKYNGENIICTLTDKRNALKEHVGLDFIFNYNRAAGYCPISEVLEGRNDRIKRFYWKVWFGNEPYPEEASITDTFTGPEVEITGSMIDDFCRVVGNQNEVYTKRGNAKRTAPMDFAIVVGWQAITKAIFPKTIDGDLLRLVHLSNAFRMLGETPLREGDKVKTSAVITAVINRENGKMVEVKGTLSANGTDVLEVVTRFLYRGNFTDFENTFEHTQETPMELTLSSPKDVAVLQSKAWFQVLDPERDLSGASLTFRLNSYSRFKNKTIRASVQTKGVVLMELPTKEVIQVASVDFQSVDCHGNPVMEFLKRQGKPIEQPVEFENGGYSLIRIMDDAYSSTFVTPPTNTPYAEVSGDYNPIHVSSVFAAFVQLPGVHGITHGMFNSAATRRFVETFAAQNVPDRVKQYEVKFVNMALPNSELVTKLSHVGMINGRKIIKIQVVNKETEEVVIVGSAEVEQPVTAYVFTGQGSQEQNMGMDLYSTSPVARAIWDSADNHFLSNFGFSILDIVRNNPKNLTIHFGAAKGQFIRKNYMDMIYEKANEDGTTSIVPVFPTITEESTSYTFTHPQGLLSATQFTQPALTLMEKAAFEDMRSRGLVQQNSVFAGHSLGEYSALSSMGDVMTIEDLVDLVFLRGLTMQNAVHRDAEGRSEYGMVAASPLRVSPTFNEAGLRFIVDHIAQQLKLLLEIVNFNVENQQYVVSGHLHSLSTMGNVLNFLKVQKIDFQKLVETLSMEQLKAELSEIVSKCHEKTLEELKKNNGIIELQRGYATIPLKIDVPFHSTFLRGGVRMFREYLMKRITPHQVNISKLRHKFIPNLTARPFDVTKEYFQEVYELTGSQRIKKIIQNWDTYASS

SEQ ID NO: 21 - protein, 1841 aa, synthetic construct
MRPEVEQELAHTLLLELLAYQFASPVRWIETQDVLLSPPYSSERLVEIGPSPTLAGMAQRTLKMKYENIDAALSMNREVLCYSRDKKNIYYNIENEEPQEVAPEPVAAAAPTPAAAAPAAPAAAPAPAASEPVAAAAELPDELPKAVDVLHILVAQKLKKTVDEVSPQKAIKDLVGGKSTLQNEILGDLQKEFGSTPEKPEETPLDELGATMQTSFSGQLGKQSSSLISRMISSKMPGGFSNSAVRAYLKNRFGLATGRTNSVLLLGLTMEPAARIGSEAEAKAWLDTVAQKYASRNGITLSAPAAASSSGGGQAAVIDEATFKKLTQSNTKLVTQQLELFANYLNKDLRAGDKAAVAERAVSDALRAQLDLWNNEHGEFYASGISPIFSPLKARVYDSEWNWARQDALKMFYDIIFGRLKLVDREIVSRCIAVMNRANPVLLEFMQYHIDHCPSDKGATYQLAKELGQQLIDNCKEAIEKPPVFKDVNYPTAPSTEIDERGNLNYEEVPRSGVRKLEQYVAEMARGGKVPPASKSKAKVQDDLARIYRIITSQNKMTRSSKLQVKQLYAQVLRNLNIRLPSGNDKTPAKETIPFLHLCRKSGEAWEFNKTLTAAYLDVLENGAKNGITFHGKCALVTGAGAGSIGVQIVKGLLAGGARVIVTTSRYSRKVTEFYQSLFTRYGSRGSSLIVVPFNQASKQDVEALVKYIFDEKDGLGWNLDYIVPFAAIPENGREIDNIDSRSELAHRIMLTNVMRLLGAVKVEKAARGFDTRPAQVILPLSPNHGTFGNDGMYSESKLALEALFNRWSSESWSNYLLICGAVIGWTRGTGLMAPNNIVAQGIEKYGVRTFSQAEMAFNILGLMSQKIVNICQTEPVYANLNGGLELLPSIKDLSTRLRTELIQTAEIRKAVAAEAAFDHNVLNGPGSDAIYHKTAIQPRANLKFEFPKAKPYEAYAHLSHLRGMVDLERVAVVTGFSEVGPWGNSRTRWDMECFGEFSLEGCVELAWIMGLIKNFNGTLKNGKPYSGWVDAKTGEPVDDKDVKSKYESYMIEHCGIRIVESDMFNDYHPEKKELLQEVVIDHDLEPFEASKDAAHEFKLKHGDKVEIFAIPDSTEWSVRFKRGTSLLVPKALRFDRFVAGQIPRGWDPKRYGIPEDIVSQVDPTTLYVLVSTVEALVSSGITDPYECYKYIHVSELGNAVGSGVGGMSALRGMYKDRWCDKPVQKDILQESFINSASAWINMLLLSSSGPIKTPVGACATAVESVDAAVEMISSGKAKICIAGGFDDFSEEGSYEFANMGATSNSIKETERGRTPAEMSRPATTTRDGFMEAQGAGVEIVMQAKLAIEMGVPIYGIIGYANTAMDKQGRSVPAPGKGVLTGARELQSKFPSPLLDIKYRARQLKLRRQQISDWAEQEYLYLDEELSAIKSQDPSVNIDDYRLERVAHIKKEIARQDKEALATFGNDFWRKDPSIAPIRGALAVWGLGVDDIGVASFHGTSTVANEKNECDVLDSQLKHLNRSKGNPILGVFQKYLTGHSKGGAGAWMLNGVLQILQTGYVPGNRNADNIDAYMERFDHVMFPSQGIQTDGVRAGSVTAFGFGQVGGQIIAIHPDYVLATLDKDTYEAYIAKTKIRYQASYRYTHDALVNNNLFRAKDHPPYTAEQEKIVYLNPEARAYLDEKTNTYKYASGKPKPKKQPASVSKTASMLDSMTKALAGSSQTVGVDVELIAAINVSNDTFLERNFTEAERSYCSSTPNPQASYAGRWSAKEAVFKSLGVKGQGAGASLKDIEIVASESGAPTVQLHGAAKEAADNAGVKNVSVSISHDDIQSVSVAIAEH

SEQ ID NO: 22 - protein, 545 aa, synthetic construct
MTASSALETSLPHSVGPEAATTTAKPPRAPLRMRHPDYTQTDVLDSSDSDAASDSEGETTAVDDGTYEDDNYVRKVLSKEKPLPPITWKNIHRNIQWISTLALTIVPLLAIYGAFTTPLKWQTAVWSVVYYYYTGLGITAGYHRLWAHRSYTASLPLQYFLALGGSGAVEGSVKWWSRGHRAHHRYTDTDLDPYSAQKGFWWAHLGWMIVKPRRRPGVADVSDLNNNPVVKWQHRYYLPLILGMGFVFPTIVAGLGWGDFRGGFFFAGAARLLFVHHSTFCVNSLAHWLGETPFDDKHTPKDHWLTALATVGEGYHNFHHEFPSDYRNALRWWQYDPTKLFIWTMSKLGLASQLKTFPDNEIKKGQYAMTLKAVAREAENIEWPKSSNHLPVLTWDEFQDACKTRQLLVVAGFIHDVSTFIDQHPGGAGLIKTRLGRDATTAFYGGYYDHSNGAANLLAQYRVGVIEGGYEVEHMKKYSEVVENLKKHGADGVAGKSADLVKGPKQTSVIKGDPQLKSAPLETLAKPPTFSETNLLGGLSLKVKA

SEQ ID NO: 23 - protein, 517 aa, Rhodotorula graminis
MRHPDYSQGDVVESSDSDREASDSEGETTAVDDGTYQDDNFVRKVLAKERPLPPITLKTLPQNINVISTLALTVVPALAIYGAFTTQIKWQTALWSVIYYFYTGLGITAGYHRLWAHRSYTASLPLQYFLALGGSGAVEGSIKWWSRGHRAHHRYTDTDLDPYSAQKGFWWSHIGWMVVKPRRTPGVADVSDLSVNEVVKWQHRWYVYLIVGMGFVFPTLVAGLGWGDYRGGFFFAGAARLLFVHHSTFCVNSLAHWLGETPFDDKHSPRDHWITALVTVGEGYHNFHHEFPQDFRNAIQTFQYDPTKWFIIVMHWLGLASQLKTFPDNEIRRGQYAMKLKAVAREADEIRWPKSSNHLPVLTWDEFQEACKTRQLMVISGYIHDVSTFIDEHPGGRALIKTRLGRDATNAFYGGYYDHSNGANNVLAQYRVGVIEGGYEVEHLKRFSKLIEDLKESGADGVAGKSADLHGGAKSSTRTTVIKGDPQLKSAPLADVAQPPTFKNVRLTGGLGHAIYA

SEQ ID NO: 24 - protein, 551 aa, synthetic construct
MAAAQPTTAVNAALATALPHAVDSATTTAKPPRAPLRMRHPNYAEGDVVESSDSDREATDSEGETTAVDDGTYQDDNYVRKVLAKERPLPPITLKTLPQNINVISTLALTVVPALAIYGAFTTQIRWQTALWSVIYYFYTGLGITAGYHRLWAHRSYTASLPLQYFLALGGSGAVEGSIKWWSRGHRAHHRYTDTDLDPYSAQKGFWWSHIGWMIVKPRRTPGVADVSDLSANEVVKWQHRWYVYLIVGMGFVFPTLVAGLGWGDYRGGFFFAGAARLLFVHHSTFCVNSLAHWLGETPFDDKHSPRDHWITALVTVGEGYHNFHHEFPQDFRNAIQTFQYDPTKWFIIVMHWLGLASQLKTFPDNEIRRGQYAMKLKAVAKEADEIRWPKDSNHLPVLTWDEFQEACKTRQLMVISGYIHDVSTFIDEHPGGRALIKTRLGRDATNAFYGGYYDHSNGANNVLAQYRVGVIEGGYEVEHLKRFSKLIEDLKESGADGVAGKNADLQSGPARVTVIKGDPQLKSAPLADVAKPPTFKNVRLTGGLGHPIEA

SEQ ID NO: 25 - protein, 478 aa, synthetic construct
MAMAAEHAVTSALATSLPHSVGPEAAAKPPRAPLRMRHPDMTNMPDSSDSDHDASDSEGETTAVDDGTYNDDNYVRKVLAKEKPLPPITLGNLYKNINVISTLALTVVPALAIYGAFTTPVMWQTALWAVAYYFYTGLGITAGYHRLWAHRAYTASLPLQYFLALGGSGAVEGSIKWWSRGHRAHHRYTDTDLDPYSASKGFWWAHVGWMIVKPRRRPGVADVSDLSVNPVVQWQHRWYLPLIVGMGFVFPTVVAGLGWGDWRGGFFFAGAARLLFVHHSTFCVNSLAHWLGETPFDDKHSPKDHWVTALVTIGEGYHNFHHEFPQDYRNATKFYQWDPTKLFIRTASLLGLASQLKTFPDNEIRRGQFAMKLKAISRQAEEIQWPKDSNHLPVLTWDDFEAECKTRQLMVIGGFIHDVSTFIDEHPGGRALIKTRLGRDATTAFYGGVYDHSNGASNVLSRYRVGVISGGYEVEHMK

SEQ ID NO: 26 - protein, 590 aa, synthetic construct
MSAAVLPMTPPLEKEESELVHRRPSPADELTPPHTPEQNPCKSTNGKGKDPIDPSLLPSDRHIPDNYVSYTIANQKYLPPITWKNLIHNIQWISFLALTVTPSLAIYGIFTTTWNTKTAIWSVIYYFITGLGITAGYHRLWAHRAYNASIPLQYVLATAGSGAVEGSIKWWCRGHRAHHRYTDTDLDPYSAEKGFFWSHVGWMLVKPRGKIGVADVSDLSKNRVVKWQHRNYIPLILGMGFVFPTVVAGLGWGDWRGGFFFAGAARLCFVHHSTFCVNSLAHWLGEQPFDNKHSPRDHIITALCTIGEGYHNFHHQFPQDFRNAIKWFQYDPTKWFIWTMSTLGLASHLKRFPDNEVKKGQYTMKLQLLKEQADELQWPKSSNDLPVISWDDFKAEAKERSLVAIHGFIHDCSSFVEDHPGGAHLIKRAIGTDATTAFFGGVYDHSNAAHNLLAMMRVGILDGGMEVEHLKRRPAESAASSIINSPVSSASASSVDIQSLADDDFRLDQTQLNSQGPKPKAPFGQPQAQVADRWTLSVPPSEKLRIVQTVPEIRPGLLTHRSVGKLDKVTKADVGGEANEFSGNEPVAAA

SEQ ID NO: 27 - protein, 594 aa, Cryptococcus neoformans
MSAAVPPMTPPFEKEASELVHRRPSPVDQLDPAFTPPHTPEQNPCKSTKGKGIDPIDPTLLASDRHIPDNYVSYTIANQKYLPPITWKNLIYNIQWISFLVLTVTPSLAIYGVFTTAWNTKTAIWSVIYYFITGLGITAGYHRLWAHRAYNAGIPLQFAFAIAGSGAVEGSIKWWCRGHRAHHRYTDTELDPYSAEKGFFWSHVGWMLVKPRGKIGVADVSDLSRNRVVKWQHRNYIPLILGMGFVVPTVVAGLGWGDWRGGFFFAGAARLCFVHHSTFCVNSLAHWLGEQPFDNKHSPRDHIITALCTIGEGYHNFHHQFPQDFRNAIKWFQYDPTKWFIWTMSQLGLASHLKRFPDNEVKKGQYTMKLQLLKEQADQLEWPKSSNDLPVISWDDFKAEAKERSLVAIHGFIHDCSSFVEDHPGGAHLIKRAIGTDATTAFFGGVYDHSNAAHNLLAMMRVGILDGGMEVEHLKRRPAESAASSVTNSPVSSASASSVDIQSLADDDFRLDQTQLNSQGPKPKAPFGQPQAQVADRWTLSVPPSEKLRIVQTVPEIRPGLLTHRQAGKLDKVTKADVGGEVIEFIGEKPVAAA

SEQ ID NO: 28 - protein, 532 aa, synthetic construct
MSAAAVSQPPPKVVNELDPEADDFQTPDNYVTKTVQNQKRLPPITWRNLLSNIQWISTLALTVPPALAIYGLMNYPLQWKTLVWSIVYYFITGLGITAGYHRLWAHRSYNASKPLQVALALAGAGSVQGSIKWWSRGHRAHHRYTDTKLDPYSAHEGFWWAHMGWMLVKPRGKIGVADISDLNKSKVVRWQHNNYVLIMVFMGLIFPTLVAGLGWGDWAGGYFFAGAARLVFVHHSTFCVNSLAHWLGETPFDNKHTPKDHFITALVTVGEGYHNFHHQFPMDFRNAIKWYQYDPTKWFIWTMSKLGLASHLKKFPDNEIKKGQYTMKLQVLQEQSKDIKWPVHSNDLPVISWEDFKAEAKERSLVAIHGFIHDVSSFIEDHPGGAHLIKKAIGTDATTAFFGGVYDHSNAAHNLLAMMRVGILDGGMEVEHLKLQGLRRNLSSASLESGVSSASSVSVQSILSEVENIENHKPEYQPVAMVSNPYTFSIPPSEKLRIIQSTPEVRPTVLSRLNSQANSAAPSRPISPPLSP

SEQ ID NO: 29 - protein, 493 aa, synthetic construct
MSASTATAPPATAPAVANPTPAAASAAAAAPAATKDKAETIDPESEHFVVSQNYVTRTVENMTMLPPVTWSNLLQNIQWISFTALTVPPAMAIYGLCTLELQRKTVIWAIVYYFMTGLGITAGYHRLWAHRAYNASAPLQYFLALCGAGSVQGSIKWWSRGHRAHHRYTDTKLDPYSAHEGFWWAHVGWMLVKPRGKIGVADISDLSRNPVVKWQHNNYVMLMVLMGLVFPTLVAGLGWGDWKGGLLFAGAARLVFVHHSTFCVNSLAHWLGETPFDNKHTPKDHFITALVTVGEGYHNFHHQFPMDFRNAIKWYQYDPTKWFIWTMSNVGLASHLKKFPDNEIKKGQYTMKLQMLQEQSGSIQWPKHSNDLPVISWEDFQAEAKERSLVAIHGFIHDCSSFLEDHPGGIHLIKKAIGTDATTAFFGGVYDHSNAAHNLLAMMRVGILDGGMEVESLKLENLQRSMSVSSMESDAASSASSVSVSSISPRWPH

SEQ ID NO: 30 - protein, 575 aa, synthetic construct
MADTAFAAMHRKAPTNPVPIPQEEKSETKLGVKPLEPTDIPSDLDVPDNYVTRTIETQKRLPPVTMANWHKNIQWVSFLALTVTPALTIYGLFNVKWNTYTAIWSVVYYFVTGLGITAGYHRLWAHRSYTASRPLEYILACAGAGAVQGSIKWWSRGHRAHHRYTDTSLDPYSAHEGFWWAHVGWMIFKPRTKIGVADISDLTRSKVVRWQHNNYLSLLLVMGLFFPMGVAGLGWGDWKGGFFFAGAARLLFVHHSTFCVNSLAHWLGEHTFDDKHTPRDHFITALVTIGEGYHNFHHQFPMDYRNAIKWNQYDPTKWFIATAQLLGLASHLKKFPDNEIKKGIYTMQLQKLAQAGEVIEWPTDSNHLPVVSWDDFVDESKNRALIAVHGFIHDVSSFMDDHPGGQHALKKFIGKDATTAFYGGVYDHSHAAQNLLAMMRVGCLEGGMEVEHLKQKVRERSLSGSSANSAFSSNEDVASLASYSSGRLSDGELSDVDFLQDLPKKEKPKHQPGCYVPDKFTLAIPPSEEFKIIRSAPRLRKTGGGHGSMASMTSLEDVSRADKIGDVAPLVAAAA